(19) United States
(12) Patent Application Publication
(10) Pub. No.: US 2025/0290020 A1
(43) Pub. Date: Sep. 18, 2025

(54) PORTABLE BIOREACTOR FOR MYCELIUM

(71) Applicants: CRAIG ELLINS, LAS VEGAS, NV (US); JOHN FRAILEY, LAS VEGAS, NV (US)

(72) Inventors: CRAIG ELLINS, LAS VEGAS, NV (US); JOHN FRAILEY, LAS VEGAS, NV (US)

(21) Appl. No.: 19/076,872

(22) Filed: Mar. 11, 2025

Related U.S. Application Data
(60) Provisional application No. 63/563,962, filed on Mar. 12, 2024.
(60) Provisional application No. 63/649,455, filed on May 20, 2024.
(60) Provisional application No. 63/704,493, filed on Oct. 7, 2024.

(51) Int. Cl. C12M 3/00; C12M 1/00; C12M 1/06; C12M 1/12; C12M 1/26; C12M 1/34; C12M 1/36

(52) U.S. Cl. CPC C12M 23/54; C12M 23/42; C12M 27/02; C12M 33/12; C12M 37/04; C12M 41/12; C12M 41/32; C12M 41/34; C12M 41/36; C12M 41/42; C12M 41/48; C12M 47/02

(57) ABSTRACT

The embodiments disclose a portable and compact bioreactor for controlled cultivation of mycelium spores, including a bioreactor cartridge with an injection port configured to transfer predetermined nutrients and mycelium spores into the bioreactor cartridge for cultivation of the mycelium spores, a pressure relief valve, a heating element, an air pump, an agitator and a cloud control system configured to analyze empirical data related to cultivation of the mycelium spores to determine the predetermined speed, the predetermined parameters and the predetermined nutrients to produce specific harvest results and to make future automatic adjustments to the predetermined speed, the predetermined parameters and the predetermined nutrients based on the specific harvest results and a bioreactor cloud data app configured to remotely monitor settings of the bioreactor, to allow the user to adjust the settings and to receive recommended settings based on the analysis of the cloud control system.

### CROSS-REFERENCED TO RELATED APPLICATIONS

This application claims priority to U.S. Provisional Patent Application No. 63/563,962, filed on Mar. 12, 2024, U.S. Provisional Patent Application No. 63/649,455, filed on Jun. 3, 2024, and U.S. Provisional Patent Application No. 63/704,493, filed on Oct. 7, 2024, the disclosures all of which are incorporated herein by reference in their entirety.

### BACKGROUND

Mushrooms are grown in soil and certain chemical components are extracted for medical and other uses from the harvested mushrooms. Despite the growing interest and demand for mushroom-derived medical products, several challenges and supply constraints persist within the market, impacting availability, accessibility, and quality. Variability in mushroom species, cultivation conditions, extraction techniques, and product formulations can result in inconsistent product quality and efficacy.

### BRIEF DESCRIPTION OF THE DRAWINGS

FIG. 1 shows for illustrative purposes only an example of bioreactor cultivation of mushrooms of one embodiment.

FIG. 2 shows a block diagram of an overview flow chart of the bioreactor cultivation of mushrooms of one embodiment.

FIG. 3 shows a block diagram of an overview of the bioreactor cultivation steps of one embodiment.

FIG. 4 shows for illustrative purposes only an example of a bioreactor cartridge of one embodiment.

FIG. 5A shows for illustrative purposes only an example of a bioreactor cartridge lid of one embodiment.

FIG. 5B shows for illustrative purposes only an example of a bioreactor cartridge magnetic motor support of one embodiment.

FIG. 6 shows for illustrative purposes only an example of a bioreactor cartridge agitator of one embodiment.

FIG. 7 shows for illustrative purposes only an example of a bioreactor cartridge motor driver of one embodiment.

FIG. 8 shows for illustrative purposes only an example of a bioreactor cartridge agitator rotor rod connection of one embodiment.

FIG. 9 shows for illustrative purposes only an example of a bioreactor cartridge heating element base of one embodiment.

FIG. 10 shows for illustrative purposes only an example of a bioreactor cartridge thermistor tube of one embodiment.

FIG. 11 shows for illustrative purposes only an example of a bioreactor system model 2 of one embodiment.

FIG. 12 shows for illustrative purposes only an example of an artificial intelligence (AI) bioreactor cloud control system of one embodiment.

FIG. 13 shows for illustrative purposes only an example of commercial and home installations of one embodiment.

FIG. 14 shows a block diagram of an overview of types of production mycelium of one embodiment.

FIG. 15 shows a block diagram of an overview of types of harvested mycelium bioactive compounds of one embodiment.

FIG. 16 shows a block diagram of an overview of bioreactor control of growth conditions of one embodiment.

FIG. 17 shows a block diagram of an overview of the extraction of mycelium bioactive compounds of one embodiment.

FIG. 18 shows for illustrative purposes only an example of the bioreactor cloud data app of one embodiment.

FIG. 19 shows for illustrative purposes only an example of a magnetic collar of one embodiment.

FIG. 20 shows for illustrative purposes only an example of an agitator rotor rod of one embodiment.

FIG. 21 shows for illustrative purposes only an example of a bioreactor cartridge controls of one embodiment.

FIG. 22 shows for illustrative purposes only an example of a control display screen of one embodiment.

FIG. 23 shows for illustrative purposes only an example of oxygenation air flow vents of one embodiment.

FIG. 24 shows for illustrative purposes only an example of a transparent view of a bioreactor of one embodiment.

FIG. 25A shows for illustrative purposes only an example of mycelium liquid culture-nutrients in a liquid solution of one embodiment.

FIG. 25B shows for illustrative purposes only an example of plating spores to culture of one embodiment.

FIG. 26 shows for illustrative purposes only an example of refillable growing medium cartridge of one embodiment.

FIG. 27A shows for illustrative purposes only an example of a syringe of one embodiment.

FIG. 27B shows for illustrative purposes only an example of syringe extracting liquid nutrient of one embodiment.

FIG. 27C shows for illustrative purposes only an example of syringe injecting best growth spores of one embodiment.

FIG. 28A shows for illustrative purposes only an example of refillable growing medium cartridge placed in bioreactor of one embodiment.

FIG. 28B shows for illustrative purposes only an example of bioreactor app of one embodiment.

### DETAILED DESCRIPTION OF THE INVENTION

In the following description, reference is made to the accompanying drawings, which form a part hereof, and which are shown by way of illustration a specific example in which the invention may be practiced. It is to be understood that other embodiments may be utilized and structural changes may be made without departing from the scope of the present invention.

It should be noted that the descriptions that follow, for example, in terms of the bioreactor mushroom cultivation and extraction for medicinal uses, devices, and methods are described for illustrative purposes and the underlying system can apply to any number and multiple types of mushrooms. In one embodiment of the present invention, the bioreactor mushroom cultivation and extraction for medicinal uses, devices, and methods can be configured using multiple hardware systems for cultivation and extraction. The bioreactor mushroom cultivation and extraction for medicinal uses, devices, and methods can be configured to include a home-sized bioreactor-based cultivation system and can be configured to include large-sized commercial mass production bioreactor-based cultivation and extraction systems using the present invention.

One of the primary challenges facing the market for mushroom-derived medical products is the lack of standardized extraction methods and quality control measures. Without standardized protocols and rigorous quality assurance processes, ensuring the safety, potency, and reliability of mushroom-derived medical products becomes challenging for manufacturers and consumers alike.

There is a growing body of scientific research supporting the therapeutic potential of mushroom-derived bioactive compounds. The supply chain for mushroom-derived medical products is susceptible to disruptions, vulnerabilities, and seasonality factors. Dependence on wild-harvested mushrooms or limited cultivation capacities for specific mushroom species may result in supply shortages, price fluctuations, and market volatility.

Mushroom-derived medical products, particularly those containing specialized extracts or proprietary formulations, may be costly and inaccessible to certain consumer demographics due to affordability concerns. High production costs, research and development expenses, and regulatory compliance requirements contribute to elevated product prices, limiting affordability and equitable access to mushroom-based healthcare solutions for underserved populations.

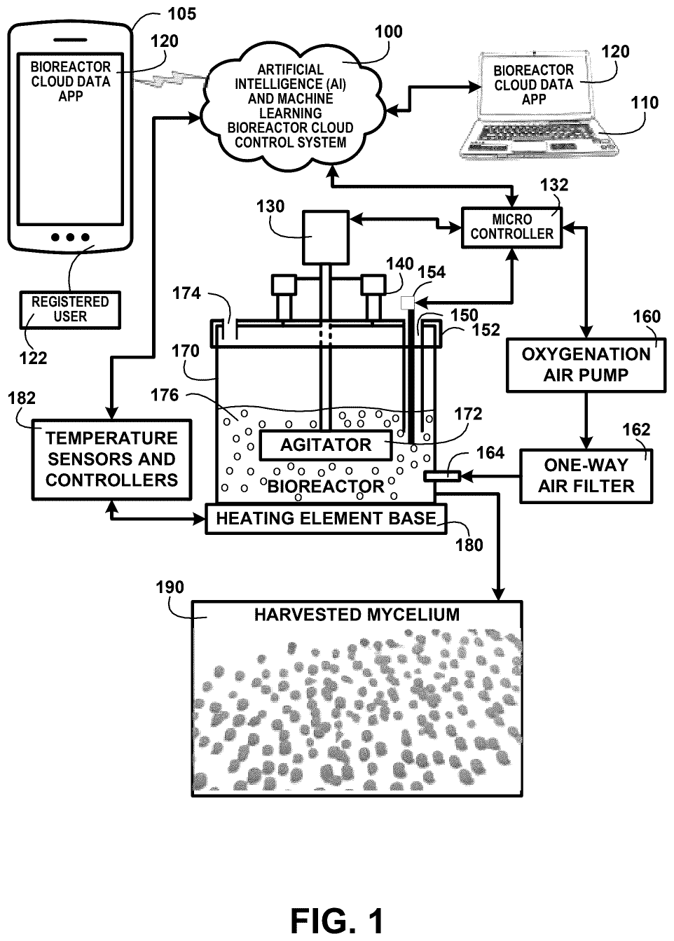

FIG. 1 shows for illustrative purposes only an example of bioreactor cultivation of mushrooms of one embodiment. FIG. 1 shows an artificial intelligence (AI) and machine learning bioreactor cloud control system 100 wirelessly coupled to a computer 110 having a bioreactor cloud data app 120. A bioreactor is a system to regulate the culturing of biological organisms in a controlled environment. A microcontroller 132 coupled to the artificial intelligence (AI) and machine learning bioreactor cloud control system 100 regulates the operations of at least a motor driver 130 coupled to a magnetic motor support 140.

A registered user 122 mobile device 105 with the bioreactor cloud data app 120 allows the registered user 122 to remotely check on the settings of the bioreactor cultivation system. The registered user 122 receives recommended settings from the artificial intelligence bioreactor cloud control system 100 and can adjust the settings using the bioreactor cloud data app 120.

The microcontroller 132 also receives temperature readings of the cultivation from a thermistor 154 inserted through a thermistor tube 150 integrated into a lid 152 of a bioreactor cartridge. An oxygenation air pump 160 pumps an airflow through a one-way air filter 162 into an oxygenation portal 164 integrated into the bioreactor cartridge to supply oxygen to the spores being cultivated.

The bioreactor cartridge 170 is equipped with an agitator 172 coupled to the motor driver 130 to rotate the cultivation and improve cultivation with agitation.

A one-way spores injection port 174 integrated into lid 152 provides access to inject mushroom spores inoculated sterile substrate into the bioreactor cartridge 170 to begin the controlled environment cultivation of mushrooms. Feed and sampling ports coupled to bioreactors feature ports for introducing nutrient feeds, substrates, or gases into the system and for withdrawing samples for analysis or monitoring.

The growing medium substrate with suspended mushroom spores 176 is heated with a heating element base 180. The temperature of the substrate is regulated by the microcontroller based on analytical input data from the artificial intelligence (AI) and machine learning bioreactor cloud control system 100. Temperature sensors and controllers 182 are monitored by the microcontroller 132, and the rpm of the agitator and oxygen levels data is transmitted by the microcontroller 132 to the artificial intelligence (AI) and machine learning bioreactor cloud control system 100 to analyze and adjust operational settings of the components to optimize the cultivation condition. The controlled environment of the bioreactor cultivation improves the growth and quality of the spores to produce quality harvested mycelium 190 for the year-round production of one embodiment.

The bioreactor was developed for bioreactor cultivation of mushrooms. The bioreactor takes advantage of an artificial intelligence (AI) and machine learning bioreactor cloud control system 100 wirelessly coupled to a computer having a bioreactor cloud data application 120. A microcontroller in communication with the artificial intelligence (AI) and machine learning bioreactor cloud control system 100 regulates the operations of the systems. The systems provide AI recommended levels of oxygen to optimize the growth of the mycelium. Activates movement within the growing substrate to ensure each mushroom spore is in contact with nutrients within the liquid substrate and supplied oxygen. The AI controller determines which and how much of the nutrients to add to the substrate based on the analyzed success of the varieties of mycelium cultivated. The growing environment is controlled to optimize the growth conditions determined by the most successful conditions learned in the AI tracking of all bioreactor cultivations and harvests. Bioreactors reduce the growth mass of the mushroom sections being cultivated thereby facilitating the extraction of the targeted essential portions of the mushroom that contain the medicinal components. The use of the bioreactor cultivation is not subject to weather conditions and wild-grown mushroom location constraints thus leading to year-round high yield harvest production of the mycelium medicinal components.

The cutting-edge AI-powered bioreactor technology is unlocking the future of mushroom cultivation. The system combines artificial intelligence, machine learning, and cloud control to optimize every stage of mycelium growth. The AI-driven bioreactor wirelessly connects to a cloud-based app, ensuring real-time, seamless control over the cultivation process. With precision microcontroller regulation, the bioreactor delivers AI-recommended oxygen levels and ensures optimal nutrient distribution, maximizing the growth potential of each mushroom spore. The intelligent system continuously analyzes cultivation success, fine-tuning nutrient levels for superior results. The growth environment is optimized based on AI's learned data from previous harvests, ensuring perfect conditions for mycelium development. By focusing on the essential components of the mushroom, the bioreactor facilitates targeted extraction of medicinal properties, delivering maximum potency. Free from the limitations of weather and wild-grown mushroom locations, the bioreactor system guarantees year-round, high-yield production of mycelium with enhanced medicinal benefits.

The current invention solves many of the problems confronting the mushroom-based medical products market. The use of bioreactor cultivation is not subject to weather conditions and wild-grown mushroom location constraints. Harvesting wild-grown and full plant cultivation is labor intensive. Bioreactors reduce the growth mass of the mushroom sections being cultivated thereby cutting the bio-waste and facilitating the extraction to the targeted essential portions of the mushroom that contain the medicinal components.

Cultured mushroom mycelium in the bioreactors creates a significant improvement in harnessing the medicinal benefits of mushrooms. This innovative approach allows for the controlled growth of mycelium in optimized conditions, leading to enhanced production of bioactive compounds with significant medicinal potential. The cultivation of mushroom mycelium in bioreactors and the extraction of bioactive compounds, along with their potential medicinal benefits starts with the cultivation of mushroom mycelium in bioreactors. The bioreactors provide a controlled environment for the growth of mushroom mycelium, offering precise control over factors such as temperature, pH, oxygen levels, and nutrient availability. This enables optimization of the mycelial growth and maximizes the production of desired bioactive compounds.

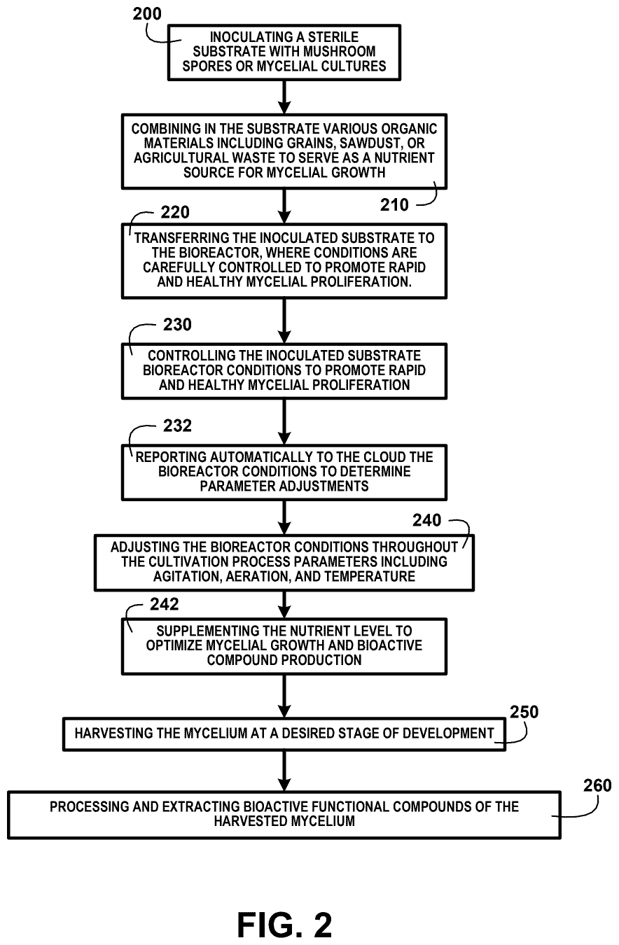

FIG. 2 shows a block diagram of an overview flow chart of the bioreactor cultivation of mushrooms of one embodiment. FIG. 2 shows inoculating a sterile substrate with mushroom spores or mycelial cultures 200. The mushroom spores are fed nutrients by combining the substrate with organic materials including grains, sawdust, or agricultural waste to serve as a nutrient source for mycelial growth 210. Reaching a predetermined growth the process continues by transferring the inoculated substrate to the bioreactor, where conditions are carefully controlled to promote rapid and healthy mycelial proliferation 220. Continual monitoring and controlling component operations are done for the inoculated substrate bioreactor conditions to promote rapid and healthy mycelial proliferation 230. The control includes reporting automatically to the cloud the bioreactor conditions to determine parameter adjustments 232. Adjusting the bioreactor conditions throughout the cultivation process parameters including agitation, aeration, and temperature 240 improves cultivation results. The spores grow well by supplementing the nutrient level to optimize mycelial growth and bioactive compound production 242. The controlled bioreactor cultivation results in harvesting the mycelium at a desired stage of development 250. Harvesting is followed by processing and extracting bioactive functional compounds of the harvested mycelium 260 of one embodiment.

The bioactive compounds extracted from the bioreactor cultured mycelium include Polysaccharides. Mushroom mycelium cultivated in bioreactors is rich in polysaccharides, and complex carbohydrates with diverse physiological effects. Beta-glucans, a type of polysaccharide found in many mushroom species, exhibit immunomodulatory properties, and have been studied for their potential to boost immune function and combat diseases.

Cultivating mushroom mycelium in the bioreactors represents an innovative approach to harnessing nature's pharmacy and unlocking the therapeutic potential of bioactive compounds. By optimizing conditions for mycelial growth and extraction the production of high-quality extracts rich in polysaccharides, triterpenoids, and other valuable compounds is increased in volume and quality. The optimization includes proprietary formulation of bioreactor substrates including nutrients formulated to promote the cultured mushroom mycelium growth and quality. Additionally, optimization includes proprietary formulation of extraction liquids to increase the quantity and quality of the extracted bioactive compounds.

These bioactive compounds offer a wide range of medicinal benefits, from immune modulation and anti-inflammatory effects to antioxidant activity. The bioreactor technology of this invention improves the development of novel therapeutics and nutraceuticals derived from cultured mushroom mycelium.

Mushroom Mycelium is cultured in the bioreactors as a source of medicinal bioactive compounds. Mushroom mycelium, the intricate network of thread-like structures that form the vegetative part of fungi, holds remarkable potential as a source of bioactive compounds with medicinal benefits. Mycelium, often referred to as the “hidden half” of fungi, plays a crucial role in nutrient cycling, soil health, and ecosystem resilience. Beyond its ecological significance, mycelium harbors a treasure trove of bioactive molecules that have been studied for their therapeutic properties.

Bioreactor culture mushroom mycelium represents a natural source of medicinal products, offering a rich source of bioactive compounds with diverse medicinal properties. From immune support and anti-inflammatory effects to antioxidant activity benefits, bioreactor cultures of mycelium-derived compounds hold promise for addressing a wide range of health concerns. The therapeutic potential of mushroom mycelium may lead to the development of novel treatments and supplements that promote wellness and vitality. Mycelium, as the vegetative part of fungi, is a versatile and functional organism with numerous applications, particularly in the realm of health and wellness.

Medicinal Properties: Mycelium produces a wide array of bioactive compounds, including polysaccharides, terpenoids, and phenolic compounds, which have been studied for their medicinal properties. These compounds exhibit biological activities such as anti-inflammatory, antioxidant, immunomodulatory, and antimicrobial effects.

Nutritional Benefits: Some species of mushrooms grown from mycelium are not only delicious but also highly nutritious. They are rich sources of protein, dietary fiber, vitamins (such as B vitamins and vitamin D), and minerals (such as selenium and potassium). Incorporating these mushrooms into diets can help improve overall nutrition and contribute to better health outcomes. Gut Health: Mycelium-derived products, particularly those containing prebiotic fibers, can support gut health by promoting the growth of beneficial gut bacteria. These fibers serve as food for probiotics, helping to maintain a healthy balance of microorganisms in the gut microbiome. A balanced gut microbiome is associated with improved digestion, enhanced immune function, and reduced risk of gastrointestinal disorders.

Mushroom Mycelium is cultured in the bioreactors as a source of medicinal bioactive compounds. Mushroom mycelium, the intricate network of thread-like structures that form the vegetative part of fungi, holds remarkable potential as a source of bioactive compounds with medicinal benefits. Mycelium, often referred to as the “hidden half” of fungi, plays a crucial role in nutrient cycling, soil health, and ecosystem resilience. Beyond its ecological significance, mycelium harbors bioactive molecules that have been studied for their therapeutic properties and the potential medicinal benefits they offer.

Bioactive Compounds Found in Mushroom Mycelium include Beta-Glucans: Beta-glucans are polysaccharides found abundantly in mushroom mycelium. These compounds possess immunomodulatory properties, stimulating the activity of macrophages, natural killer cells, and other components of the immune system. Beta-glucans have been studied for their potential in enhancing immune function and combating diseases, including cancer, infections, and autoimmune disorders.

Polysaccharide Peptides: Certain mushroom mycelium species produce polysaccharide peptides, complex molecules with potent antioxidant and anti-inflammatory properties. These compounds have shown promise in protecting cells from oxidative damage, reducing inflammation, and supporting overall health and well-being.

Triterpenoids: Triterpenoids are another class of bioactive compounds found in mushroom mycelium. These molecules exhibit diverse pharmacological activities, including anti-inflammatory, antimicrobial, and anti-cancer properties. Triterpenoids have been studied for their potential to manage chronic inflammatory conditions, prevent infections, and inhibit tumor growth.

Aromatic Compounds: Mushroom mycelium is rich in aromatic compounds, such as phenols and flavonoids, which contribute to its distinctive aroma and flavor. These compounds possess antioxidant properties and may help protect cells from oxidative stress and DNA damage. Additionally, some aromatic compounds found in mycelium have demonstrated antimicrobial activity against pathogens.

Medicinal benefits of mycelium-derived bioactive compounds include immune support with Beta-glucans and other immunomodulatory compounds found in mushroom mycelium have been shown to enhance immune function. By activating immune cells and promoting cytokine production, these compounds help the body mount a robust immune response against infections, tumors, and other threats.

Anti-Inflammatory Effects: Polysaccharide peptides and triterpenoids present in mushroom mycelium exhibit potent anti-inflammatory properties. These compounds help mitigate inflammation by suppressing the production of pro-inflammatory cytokines and inhibiting inflammatory pathways. As a result, mycelium-derived bioactive compounds may benefit individuals suffering from chronic inflammatory conditions such as arthritis, inflammatory bowel disease, and asthma.

Antioxidant Activity: Aromatic compounds and polysaccharide peptides found in mushroom mycelium possess significant antioxidant activity. By scavenging free radicals and reducing oxidative stress, these compounds help protect cells from damage and slow down the aging process. Antioxidant-rich mycelium extracts may therefore contribute to overall health and longevity.

Bioreactor culture mushroom mycelium represents a natural source of medicinal products, offering a rich source of bioactive compounds with diverse medicinal properties. From immune support and anti-inflammatory effects to antioxidant activity benefits, bioreactor cultures of mycelium-derived compounds hold promise for addressing a wide range of health concerns. The therapeutic potential of mushroom mycelium may lead to the development of novel treatments and supplements that promote wellness and vitality.

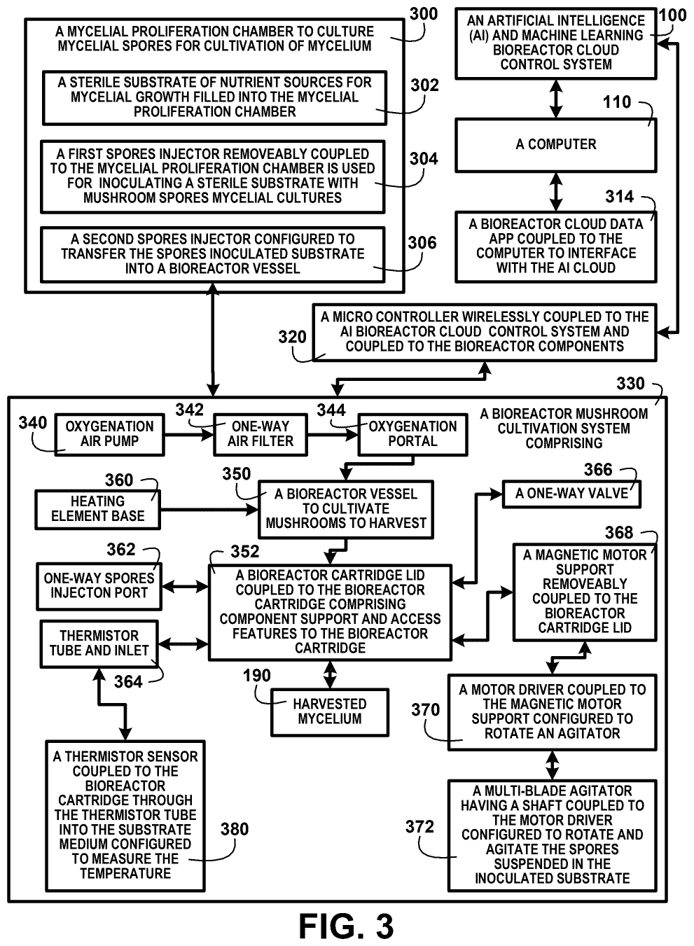

FIG. 3 shows a block diagram of an overview of the bioreactor cultivation steps of one embodiment. FIG. 3 shows a mycelial proliferation chamber to culture mycelial spores for cultivation of mycelium 300. The cultivation includes a sterile substrate of nutrient sources for mycelial growth filled into the mycelial proliferation chamber 302. A first spores injector removably coupled to the mycelial proliferation chamber is used for inoculating the sterile substrate with mushroom spores mycelial cultures 304. A second spores injector is configured to transfer the spores inoculated substrate into a bioreactor cartridge 306.

An artificial intelligence (AI) and machine learning bioreactor cloud control system 100 receive data related to the bioreactor cartridge operations from the components. A computer 110 wirelessly coupled to the artificial intelligence (AI) and machine learning bioreactor cloud control system 100 to display AI data to allow the user to assess the cultivation progress. A bioreactor cloud data app 120 coupled to the computer 110 to interface with the AI cloud 314.

A microcontroller 132 of FIG. 1 wirelessly coupled to the artificial intelligence (AI) and machine learning bioreactor cloud control system 100 and coupled to the bioreactor components 320 to transmit and receive data and instructions bidirectionally. The microcontroller 132 of FIG. 1 is coupled to the cultivation parameters components to regulate the operations.

A bioreactor mushroom cultivation system comprising 330 a plurality of components including an oxygenation air pump 340 coupled to a one-way air filter 342 that provides airflow through an oxygenation portal 344 of the bioreactor cartridge to cultivate mushrooms to harvest 350. The filtered oxygen supplies the spores with a clean air supply to aid the cultivation.

A bioreactor cartridge lid coupled to the bioreactor cartridge comprising component support and access features to the bioreactor cartridge 352 and a vent that is a one-way valve 366. Heat is generated for the spores with a heating element base 360 the bioreactor cartridge sets on. A one-way spores injection port 362 integrated into the lid provides access for the second spores injector to transfer the spores inoculated substrate to be deposited into the bioreactor cartridge. The thermistor 154 of FIG. 1 is inserted into the substrate through a thermistor tube and inlet 364 is integrated into the lid. A thermistor sensor coupled to the bioreactor cartridge through the thermistor tube into the substrate medium configured to measure the temperature 380 and transmit the temperature reading to the microcontroller 132 of FIG. 1. The thermistor 154 of FIG. 1 measures the temperature in the bioreactor cartridge.

Magnetic motor support removably coupled to the bioreactor cartridge lid 368 supports a motor driver coupled to the magnetic motor support configured to rotate an agitator 370. A multi-blade agitator having a shaft coupled to the motor driver configured to rotate and agitate the spores suspended in the inoculated substrate 372. The controlled environment created by the bioreactor cartridge and component allows improved cultivation of a plurality of mushroom species. The controlled environment assures harvested mycelium 190 of predeterminable quality of one embodiment.

The bioactive compounds extracted from the bioreactor cultured mycelium include Polysaccharides. Mushroom mycelium cultivated in bioreactors is rich in polysaccharides, and complex carbohydrates with diverse physiological effects. Beta-glucans, a type of polysaccharide found in many mushroom species, exhibit immunomodulatory properties, and have been studied for their potential to boost immune function and combat diseases of one embodiment.

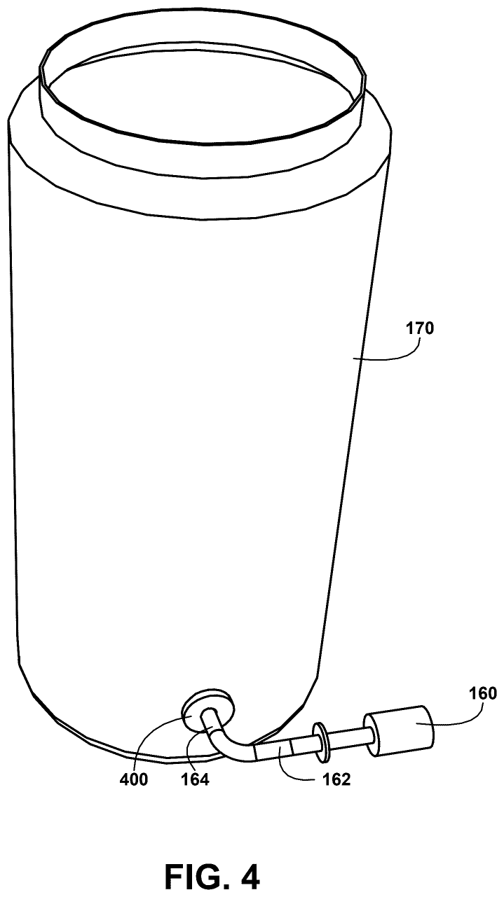

FIG. 4 shows for illustrative purposes only an example of a bioreactor cartridge of one embodiment. FIG. 4 shows that bioreactor cartridge 170, is made of at least one material from a group of at least stainless steel, glass, or plastic and comes in assorted sizes ranging from laboratory-scale to industrial-scale reactors. Oxygenation promotes the growth of the spores. An oxygenation air pump 160 is regulated to supply oxygen through the one-way air filter 162 to purify the airflow. The airflow enters the bioreactor chamber through the oxygenation portal 164 sealed with a gasket 400 of one embodiment.

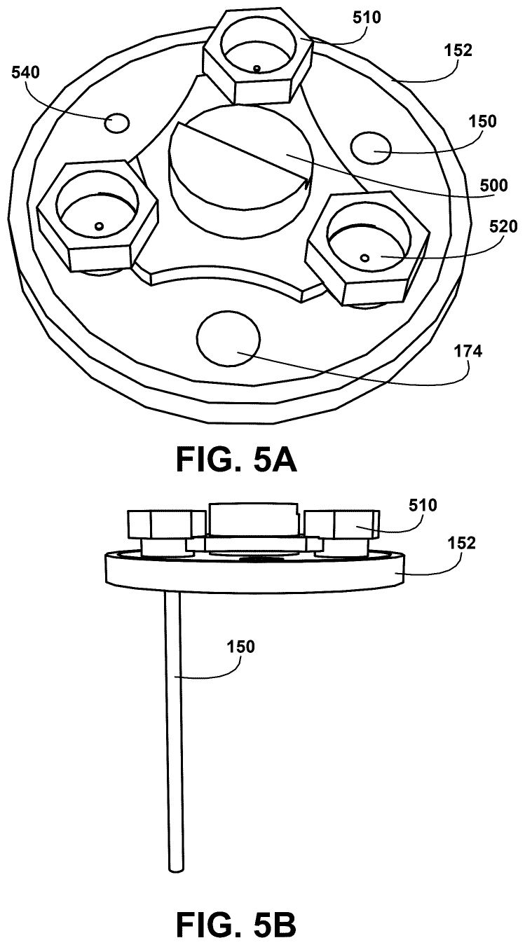

FIG. 5A shows for illustrative purposes only an example of a bioreactor cartridge lid of one embodiment. FIG. 5A shows the lid 152 of the bioreactor cartridge 170 of FIG. 1. The magnetic motor support 140 of FIG. 1 is seated into the couplings 510 and integrated into the lid 152. The couplings 510 include a magnet 520 to secure the magnetic motor support 140 of FIG. 1 to the lid 152. A drive motor coupling 500 mates with the motor driver 130 of FIG. 1 axle that has double bearings. The thermistor tube 150 extends from a lid inlet downward into the cartridge to allow the thermistor 154 of FIG. 1 to reach into the substrate-growing solution to monitor the temperature. Integrated into the lid 152 is the one-way spores injection port 174 providing access into the cartridge for depositing the substrate and spores. A one-way valve 540 integrated into the lid 152 provides a pressure relief outlet to prevent over-pressuring the bioreactor cartridge of one embodiment.

FIG. 5B shows for illustrative purposes only an example of a bioreactor cartridge magnetic motor support of one embodiment. FIG. 5B shows the lid 152 in a side view with the couplings 510. Also showing is the thermistor tube 150 extending downward from the lid 152 to allow the inserted thermistor 154 of FIG. 1 terminus to be immersed into the substrate-growing medium of one embodiment.

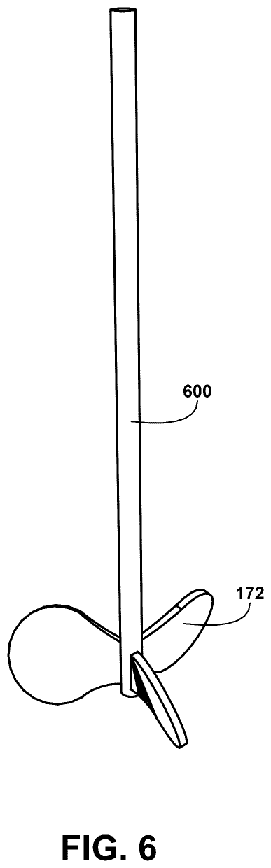

FIG. 6 shows for illustrative purposes only an example of a bioreactor cartridge agitator of one embodiment. FIG. 6 shows the agitator 172 coupled to the agitator rotor rod 600. The agitator rotor rod 600 couples to the motor driver 130 of FIG. 1 axle connector to rotate the agitator 172 to provide agitation to the substrate and spores to provide a uniform mix of the nutrients, oxygen, and temperature to all of the spores. Monitoring of the conditions is performed to make adjustments in rotational speed to maintain optimized growing conditions of one embodiment.

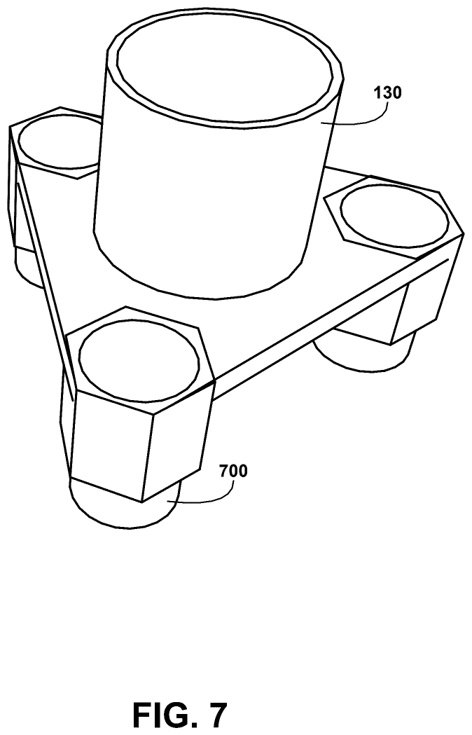

FIG. 7 shows for illustrative purposes only an example of a bioreactor cartridge motor driver of one embodiment. FIG. 7 shows the motor driver 130 coupled to the magnetic support 700. The motor driver 130 is coupled to the microcontroller 132 of FIG. 1 for a power connection and rpm regulation. The magnetic support 700 couples to the lid 152 of FIG. 1 and magnet 520 of FIG. 5A in the couplings 510 of FIG. 5A to secure the motor driver 130 of FIG. 1 in a stable connection of one embodiment.

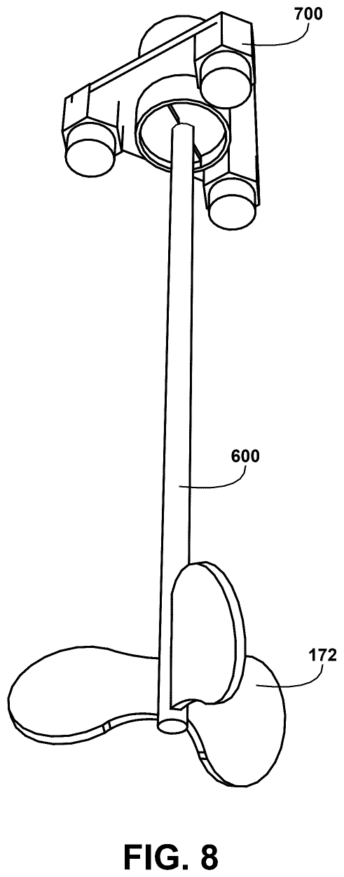

FIG. 8 shows for illustrative purposes only an example of a bioreactor cartridge agitator rotor rod connection of one embodiment. FIG. 8 shows the magnetic support 700 and agitator rotor rod 600 coupled to the motor driver 130 of FIG. 1. The motor driver 130 of FIG. 1 rotates the agitator rotor rod 600 at an rpm regulated by the microcontroller 132 of FIG. 1. The agitator 172 rotated by the agitator rotor rod 600 is continuously mixing the substrate to assure all of the suspended spores are provided nutrients, are fully oxygenated, and kept at an optimal temperature. The agitator 172 fluid movement does not harm the mycelium, and the liquid movement also imparts the mycelium marble shapes of one embodiment.

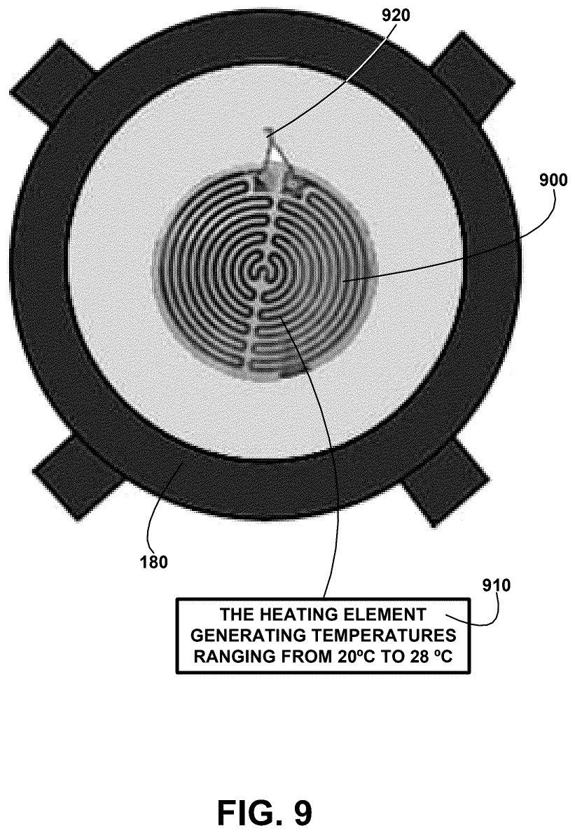

FIG. 9 shows for illustrative purposes only an example of a bioreactor cartridge heating element base of one embodiment. FIG. 9 shows the bioreactor cartridge 170 of FIG. 1 is placed to receive heat to maintain an optimal growing temperature. The heating element base 180 includes the outer support ring for the heating element 900. The heating element generates temperatures ranging from 20° C. to 28° C. 910. The heating element 900 is coupled to control and power conductors 920 to energize elements and maintain the regulated temperature settings from the microcontroller 132 of FIG. 1 as determined by the artificial intelligence (AI) and machine learning bioreactor cloud control system 100 of FIG. 1 in one embodiment. Temperature range approx. 28 c throughout most of the growth and lowered to 25c as low as 20c after the 5th day to accelerate Psilocybin content.

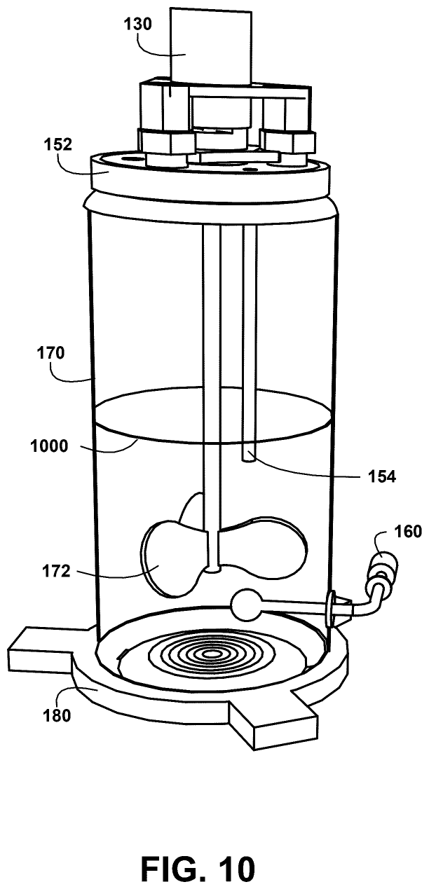

FIG. 10 shows for illustrative purposes only an example of a bioreactor cartridge thermistor tube of one embodiment. FIG. 10 shows the assembled bioreactor cartridge 170 including the motor driver 130, lid 152, oxygenation air pump 160, and heating element base 180. FIG. 10 also shows the interior of the bioreactor cartridge 170 is a substrate level 1000 into which the thermistor tube 150 of FIG. 1 is extended for inserting the thermistor 154 to measure the temperature. The agitator 172 is shown immersed in the substrate to mix the substrate for uniformity of the contents including supplemental nutrients when they are added to the substrate, temperature, and oxygen levels of one embodiment.

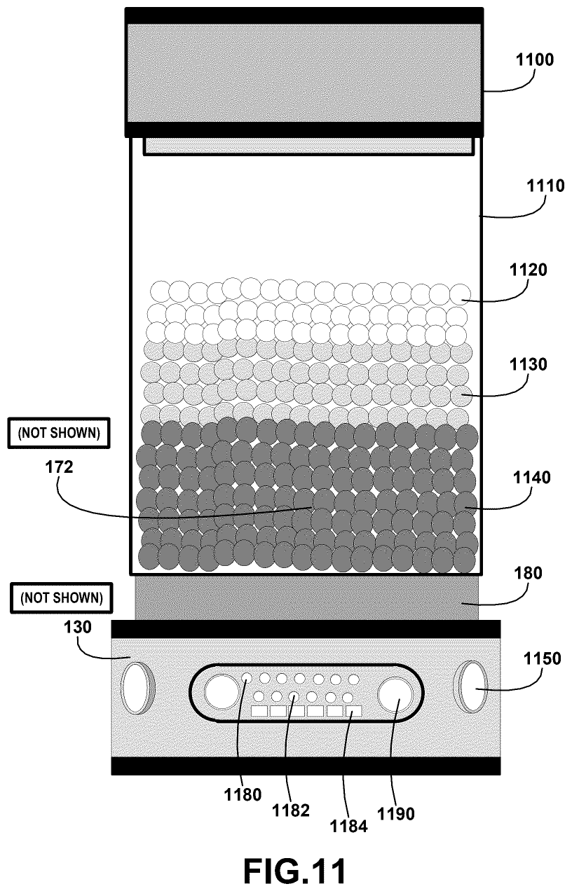

FIG. 11 shows for illustrative purposes only an example of a bioreactor system model 2 of one embodiment. FIG. 11 shows bioreactor system model 2 including a lid cover 1100, and a bioreactor 1110 chamber. The bioreactor 1110 chamber contains oxygenation bubbles 1120, substrate 1130 in bubbles, and mycelium 1140 shaped in pellets by the agitator motion. Bioreactor system model 2 also includes an agitator (not shown) 172, a heating element base 180, air pump inlet 1150 to draw air into the air pump, a motor driver (not shown) 130 to rotate the agitator, LED status lights 1180 to inform the user of the operational status, system settings status 1182 illuminated buttons, operation buttons 1184 to show red, yellow, and green signals of component status, and a motor driver on/off 1190 switch of one embodiment.

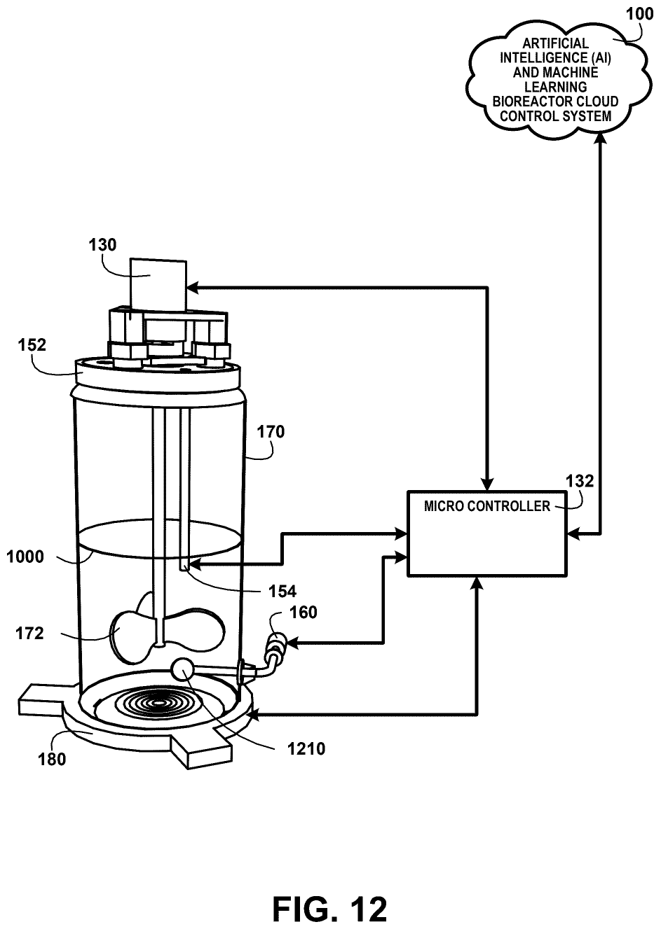

FIG. 12 shows for illustrative purposes only an example of an artificial intelligence (AI) bioreactor cloud control system of one embodiment. FIG. 12 shows the assembled bioreactor cartridge 170 including the motor driver 130, lid 152, and oxygenation air pump 160 through an air stone 1210 to create microbubbles ranging from 0.5 μm to 5 μm, heating element base 180. FIG. 12 also shows the interior of the bioreactor cartridge 170 is a substrate level 1000 into which the thermistor tube 150 of FIG. 1 is extended for inserting the thermistor 154 to measure the temperature. The agitator 172 is shown immersed in the substrate.

The assembled bioreactor cartridge 170 is coupled to the microcontroller 132 which is wirelessly coupled to the artificial intelligence (AI) and machine learning bioreactor cloud control system 100. The bioreactor conditions monitoring data received by the microcontroller 132 is transmitted to the artificial intelligence (AI) and machine learning bioreactor cloud control system 100. The artificial intelligence (AI) and machine learning bioreactor cloud control system 100 analyze the data to compare to successful previous cultivations to determine any adjustments to regulate temperature and other growing conditions to optimize the production of one embodiment.

The agitation and mixing systems include agitation mechanisms, including impellers, stirrers, or spargers for mixing nutrients, maintaining homogeneity, and preventing cell settling. The design considerations include impeller geometry, placement, and speed control to optimize mixing efficiency and minimize shear stress on cells.

The temperature control systems include precision temperature controllers for maintaining optimal growth conditions and metabolic activity. Bioreactors utilize jacketed cartridges, external heat exchangers, or immersion heaters coupled with temperature sensors and controllers to achieve precise temperature regulation. The pH and dissolved oxygen monitoring are utilized for monitoring and control of pH and dissolved oxygen levels are critical for cell viability and product yield. Bioreactors are equipped with sensors and control systems to measure and adjust pH using acid or base addition, as well as regulate oxygen concentrations through aeration or agitation speed modulation.

Quality control during manufacture is maintained to ensure the bioreactors are completed with sustainable quality to allow for proper sterilization and aseptic techniques during use without damaging the equipment and ensure sterilization and aseptic quality levels are maintainable to prevent contamination.

Automation and control systems are integrated into the advanced bioreactor manufacture. The automation and control systems, including programmable logic controllers (PLCs) and supervisory control and data acquisition (SCADA) systems, monitor and regulate process parameters, automate feeding and sampling operations, and ensure reproducibility and consistency in production.

These bioreactors and related cultivation and extraction equipment are manufactured to provide year-round cultivation. The bioreactors enable year-round cultivation of medicinal plants in a controlled environment, independent of seasonal variations and geographical constraints. This allows for continuous production and a reliable supply of plant-derived bioactive compounds, regardless of climate or location.

The optimized growth conditions provided using the bioreactors provide precise control overgrowth parameters, such as temperature, humidity, light, and nutrient supply, to create optimal conditions for plant growth, development, and metabolite production. This results in higher yields, enhanced potency, and consistent quality of medicinal plant extracts.

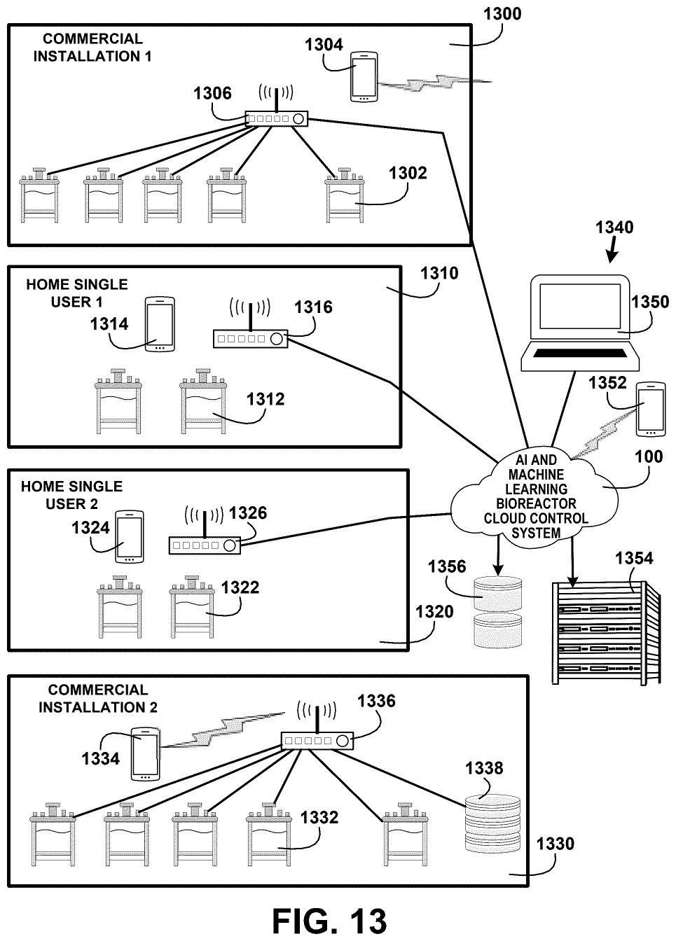

FIG. 13 shows for illustrative purposes only an example of commercial and home installations of one embodiment. FIG. 13 shows bioreactor cultivation in production for consumer home use and commercial industrial use. As shown in commercial installation 1 1300, a plurality of bioreactor systems 1302 is used to increase the total production of mycelium for extraction of bioactive compounds for use in medicinal products. The commercial operation includes a microcontroller 1306 that is coupled to bioreactors. A user mobile device having a mobile application 1304 allows the commercial personnel to be informed of each phase of each of the plurality of bioreactors systems 1302 stage of production. At least one microcontroller 132 of FIG. 1 coupled to the plurality of bioreactor systems 1302 communicates with a bioreactor cloud interface 1340 to regulate conditions in each of the plurality of bioreactor systems 1302.

Shown in a home single user 1 1310 use are, for example, two bioreactor systems 1312. A home user mobile device having a mobile application 1314 provides the home user with production data from the microcontroller 1316 via the artificial intelligence (AI) and machine learning bioreactor cloud control system 100 to keep the home user current on the growth conditions.

A home single user 2 1320 use is shown, for example, having two bioreactor systems 1322. A user mobile device having a mobile application 1324 provides production condition status to the home user from the microcontroller 1326 via the artificial intelligence (AI) and machine learning bioreactor cloud control system 100 that is wirelessly receiving determined regulation of the conditions through a bioreactor cloud interface 1340.

Commercial installation 2 1330 shows a plurality of bioreactor systems 1332 in the production of the mycelium. At least one user mobile device having a mobile application 1334 is used to receive growth conditions and regulatory adjustments of the components from the microcontroller 1336. A plurality of databases 1338 is used to store the monitoring data of the conditions.

A bioreactor cloud interface 1340 coupled to a computer having a mobile application 1350 to provide production levels on all bioreactor systems in use. A user mobile device having a mobile application 1352 allows central production monitoring personnel to determine the operating conditions of the bioreactor systems. The artificial intelligence (AI) and machine learning bioreactor cloud control system 100 is coupled to a plurality of servers 1354 and a plurality of databases 1356 of one embodiment.

Manufacturing of the bioreactors is available for large-scale production of medicinal plants in one embodiment for mushrooms. In one embodiment, the bioreactors are scaled up in size and capacity to allow higher production of the mushroom components to meet the pharmaceutical demand for the mushroom medicinal products.

Bioactive compound production from mushroom medicinal plants contains a myriad of bioactive compounds with therapeutic properties, including alkaloids, flavonoids, terpenoids, and polyphenols. Bioreactors can be engineered to stimulate the biosynthesis of specific secondary metabolites through elicitation, precursor feeding, or genetic engineering techniques, thereby enhancing the production of target compounds for pharmaceutical or nutraceutical applications.

The scalability and efficiency of the bioreactors offer scalability and efficiency advantages over traditional cultivation methods, allowing for higher plant densities, reduced land and water requirements, and increased productivity per unit area. This enables cost-effective large-scale production of medicinal plants with minimal environmental impact and resource utilization.

The extraction processes of mushroom components harness their therapeutic potential for applications, including pharmaceuticals, nutraceuticals, cosmetics, and functional foods. The diverse extraction processes employed to isolate mushroom components are used for the specific bioactive compounds and differing types of mushrooms.

Several extraction methods are utilized to isolate bioactive compounds from mushrooms, each offering unique advantages in terms of efficiency, selectivity, and scalability. Some methods include solvent extraction. Solvent extraction is one of the most widely used methods for isolating mushroom components. It involves the use of organic solvents such as ethanol, methanol, acetone, or water to dissolve and extract target compounds from mushroom biomass. Solvent extraction can be performed using conventional methods such as maceration, percolation, or Soxhlet extraction, as well as ultrasound-assisted extraction (UAE) and supercritical fluid extraction (SFE).

Supercritical fluid extraction (SFE) is a sophisticated extraction technique that utilizes supercritical fluids such as carbon dioxide (CO2) as solvents. Under high pressure and temperature conditions, CO2 exhibits both gas-like and liquid-like properties, enabling efficient extraction of non-polar and semi-polar compounds from mushrooms. SFE offers advantages such as high selectivity, minimal solvent residue, and reduced environmental impact, making it particularly suitable for extracting heat-sensitive or thermally labile bioactive compounds.

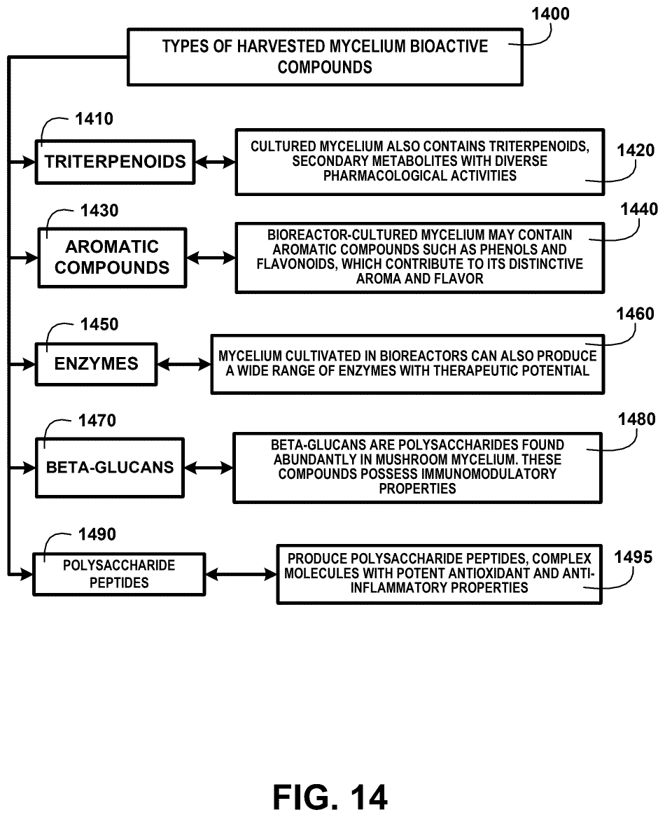

FIG. 14 shows a block diagram of an overview of types of production mycelium of one embodiment. FIG. 14 displays several types of harvested mycelium 190 of FIG. 1 bioactive compounds 1400 extracted for use in medicinal products. The mycelium bioactive compounds include triterpenoids 1410. The cultured mycelium also contains triterpenoids, secondary metabolites with diverse pharmacological activities 1420. Another type of mycelium bioactive compound is aromatic compound 1430. Aromatic compounds 1430 of the bioreactor-cultured mycelium may contain aromatic compounds such as phenols and flavonoids, which contribute to its distinctive aroma and flavor 1440.

Enzymes 1450 are bioactive compounds of the mycelium cultivated in bioreactors that can also produce a wide range of enzymes with therapeutic potential 1460. Beta-glucans 1470 are also extracted from the mycelium cultivated in bioreactors. Beta-glucans are polysaccharides found abundantly in mushroom mycelium. These compounds possess immunomodulatory properties 1480.

Mycelium cultivated in bioreactors also provides extraction of polysaccharide peptides 1490. The extractions from the mycelium cultivated in bioreactors also produce polysaccharide peptides, complex molecules with potent antioxidant and anti-inflammatory properties 1495 of one embodiment.

Triterpenoids: Cultured mushroom mycelium also contains triterpenoids, secondary metabolites with diverse pharmacological activities. These compounds exhibit anti-inflammatory, antioxidant, and anti-cancer properties, making them valuable candidates for therapeutic applications.

Aromatic Compounds: Bioreactor-cultured mycelium may contain aromatic compounds such as phenols and flavonoids, which contribute to its distinctive aroma and flavor. These compounds possess antioxidant properties and may help protect cells from oxidative stress and damage.

Enzymes: Mycelium cultivated in bioreactors can also produce a wide range of enzymes with therapeutic potential. For example, certain mushroom species are known to produce enzymes such as laccase and peroxidase, which have applications in bioremediation, food processing, and pharmaceutical manufacturing.

Medicinal benefits of bioactive compounds from bioreactor cultured mycelium include Immune Modulation. Polysaccharides extracted from cultured mycelium have been shown to modulate the immune system, enhancing the activity of immune cells, and promoting immune surveillance against pathogens. These compounds may help bolster the body's defenses and support overall health and well-being.

Anti-inflammatory effects from triterpenoids found in the bioreactor cultured mycelium exhibit potent anti-inflammatory properties, which may help alleviate symptoms of chronic inflammatory conditions such as arthritis, inflammatory bowel disease, and asthma. By suppressing inflammatory pathways and reducing oxidative stress, these compounds contribute to a balanced immune response and tissue homeostasis. Antioxidant activity is derived from aromatic compounds and polysaccharides extracted from the bioreactor cultured mycelium possess significant antioxidant activity, scavenging free radicals and protecting cells from oxidative damage. These compounds help mitigate oxidative stress and may play a role in preventing age-related diseases and promoting longevity.

Cultivating mushroom mycelium in the bioreactors represents an innovative approach to harnessing nature's pharmacy and unlocking the therapeutic potential of bioactive compounds. By optimizing conditions for mycelial growth and extraction the production of high-quality extracts rich in polysaccharides, triterpenoids, and other valuable compounds is increased in volume and quality. The optimization includes proprietary formulation of bioreactor substrates including nutrients formula to promote the cultured mushroom mycelium growth and quality. Additionally, optimization includes proprietary formulation of extraction liquids to increase the quantity and quality of the extracted bioactive compounds.

These bioactive compounds offer a wide range of medicinal benefits, from immune modulation and anti-inflammatory effects to antioxidant activity.

Mushroom Mycelium is cultured in the bioreactors as a source of medicinal bioactive compounds. Mushroom mycelium, the intricate network of thread-like structures that form the vegetative part of fungi, holds remarkable potential as a source of bioactive compounds with medicinal benefits. Mycelium, often referred to as the “hidden half” of fungi, plays a crucial role in nutrient cycling, soil health, and ecosystem resilience. Beyond its ecological significance, mycelium harbors a treasure trove of bioactive molecules that have been studied for their therapeutic properties.

Bioactive Compounds Found in Mushroom Mycelium include Beta-Glucans: Beta-glucans are polysaccharides found abundantly in mushroom mycelium. These compounds possess immunomodulatory properties, stimulating the activity of macrophages, natural killer cells, and other components of the immune system. Beta-glucans have been studied for their potential in enhancing immune function and combating diseases, including cancer, infections, and autoimmune disorders.

Polysaccharide Peptides: Certain mushroom mycelium species produce polysaccharide peptides, complex molecules with potent antioxidant and anti-inflammatory properties. These compounds have shown promise in protecting cells from oxidative damage, reducing inflammation, and supporting overall health and well-being.

Medicinal benefits of mycelium-derived bioactive compounds include immune support with Beta-glucans and other immunomodulatory compounds found in mushroom mycelium have been shown to enhance immune function. By activating immune cells and promoting cytokine production, these compounds help the body mount a robust immune response against infections, tumors, and other threats.

Antioxidant Activity: Aromatic compounds and polysaccharide peptides found in mushroom mycelium possess significant antioxidant activity. By scavenging free radicals and reducing oxidative stress, these compounds help protect cells from damage and slow down the aging process. Antioxidant-rich mycelium extracts may therefore contribute to overall health and longevity.

Bioreactor culture mushroom mycelium represents a natural source of medicinal products, offering a rich source of bioactive compounds with diverse medicinal properties. From immune support and anti-inflammatory effects to antioxidant activity benefits, bioreactor cultures of mycelium-derived compounds hold promise for addressing a wide range of health concerns. The therapeutic potential of mushroom mycelium may lead to the development of novel treatments and supplements that promote wellness and vitality.

There are medicinal benefits of psilocybin. Psilocybin, the psychoactive compound found in certain species of mushrooms, has garnered significant attention in recent years for its potential medicinal benefits. Research suggests that when administered in controlled settings, psilocybin can offer profound therapeutic effects, particularly in treating mental health disorders such as depression, anxiety, PTSD, and addiction. The following are some of the medicinal benefits of psilocybin and its legal administration.
1. Alleviation of Depression and Anxiety: Psilocybin has shown promise in alleviating symptoms of depression and anxiety.
2. Treatment of PTSD: Post-Traumatic Stress Disorder (PTSD) can be debilitating, and often resistant to conventional treatments. Psilocybin-assisted therapy has emerged as a potential breakthrough in addressing PTSD.
3. Addiction Treatment: Substance use disorders pose a significant public health challenge worldwide. Psilocybin therapy has shown promise in treating forms of addiction, including alcohol, tobacco, and opioids.
4. End-of-Life Anxiety: For individuals grappling with terminal illness, existential distress, and anxiety are common. Psilocybin therapy has demonstrated efficacy in alleviating end-of-life anxiety and improving the quality of life for terminally ill patients.

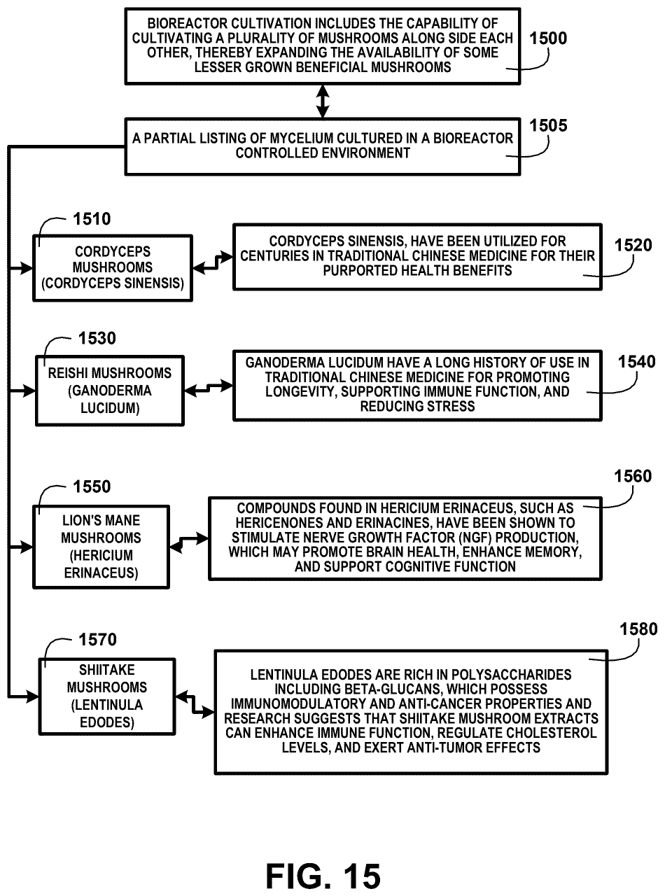

FIG. 15 shows a block diagram of an overview of types of harvested mycelium bioactive compounds of one embodiment. FIG. 15 shows bioreactor cultivation includes the capability of cultivating a plurality of mushrooms alongside each other, thereby expanding the availability of some lesser-grown beneficial mushrooms 1500. Mushrooms have long been revered in traditional medicine systems across the globe for their therapeutic properties. A partial listing of mycelium cultured in a bioreactor-controlled environment 1505 is shown as an example of the plurality of mushroom types.

Among them, Cordyceps mushrooms stand out for their remarkable health benefits and medicinal uses. In recent years, scientific research has begun to uncover the mechanisms behind these benefits, shedding light on their potential applications in modern medicine. Additionally, other mushrooms and their components have also shown promising medical properties, contributing to the growing interest in mycological medicine. A partial listing of mycelium cultured in a bioreactor-controlled environment 1505 is shown as an example of the plurality of mushroom types.

One type is cordyceps mushrooms (cordyceps sinensis) 1510. Cordyceps sinensis has been utilized for centuries in traditional Chinese medicine for its health benefits 1520. Among them, Cordyceps mushrooms stand out for their remarkable health benefits and medicinal uses. In recent years, scientific research has begun to uncover the mechanisms behind these benefits, shedding light on their potential applications in modern medicine. Additionally, other mushrooms and their components have also shown promising medical properties, contributing to the growing interest in mycological medicine.

Different mushrooms contain differing components that studies have shown are more effective in use for different medical conditions. Bioreactor cultivation includes the capability of cultivating a plurality of mushrooms alongside each other, thereby expanding the availability of some lesser-grown beneficial mushrooms.

Several types of mushrooms include the Cordyceps Mushrooms. Cordyceps mushrooms, known scientifically as Cordyceps sinensis, have been utilized for centuries in traditional Chinese medicine for their purported health benefits. These fungi grow in the mountainous regions of China, Nepal, and Tibet and have been historically used to enhance vitality, improve respiratory function, and boost immune health. One of the most well-documented benefits of Cordyceps mushrooms is their immunomodulatory effects. Studies have shown that Cordyceps extracts can enhance the activity of natural killer cells, macrophages, and other immune cells, thereby strengthening the body's defense against infections and diseases.

Chronic inflammation is implicated in diseases, including cardiovascular disorders, diabetes, and cancer. Cordyceps mushrooms contain bioactive compounds such as cordycepin and polysaccharides, which exhibit potent anti-inflammatory properties. These compounds help mitigate inflammation by inhibiting pro-inflammatory cytokines and signaling pathways.

In traditional medicine, Cordyceps mushrooms are often prescribed to improve respiratory function and alleviate symptoms of respiratory disorders such as asthma and chronic obstructive pulmonary disease (COPD). Research suggests that Cordyceps extracts can dilate bronchial passages, improve oxygen uptake, and reduce inflammation in the airways, thus offering relief to individuals with respiratory conditions.

Athletes and fitness enthusiasts have also turned to Cordyceps mushrooms for their ability to boost energy levels and enhance endurance. Cordyceps supplements are believed to improve Adenosine triphosphate (ATP) production, increase oxygen utilization, and enhance mitochondrial function, thereby promoting physical performance and stamina.

Another type is Reishi mushrooms (Ganoderma lucidum) 1530. Ganoderma lucidum has a long history of use in traditional Chinese medicine for promoting longevity, supporting immune function, and reducing stress 1540. Modern research has identified bioactive compounds in Reishi mushrooms, such as triterpenoids and polysaccharides, which exhibit antioxidant, anti-inflammatory, and immunomodulatory effects.

Yet another type is lion's mane mushrooms (Hieracium erinaceus) 1550. Compounds found in Hieracium erinaceus, such as hericenones and erinacines, have been shown to stimulate nerve growth factor (NGF) production, which may promote brain health, enhance memory, and support cognitive function 1560. Additionally, Lion's Mane mushrooms exhibit neuroprotective properties, making them a promising candidate for neurodegenerative disorders like Alzheimer's disease.

The last in this partial list is shiitake mushrooms (lenticular edodes) 1570. Lentinula edodes are rich in polysaccharides including beta-glucans, which possess immunomodulatory and anti-cancer properties and research suggests that shiitake mushroom extracts can enhance immune function, regulate cholesterol levels, and exert anti-tumor effects 1580 of one embodiment.

The invention bioreactors are versatile, scalable, and able to control parameters. These bioreactors are well-suited for use in pharmaceutical production. Bioreactors are utilized, in one embodiment, for the production of therapeutic plant-based cultivation of mushroom components for extraction in one embodiment of biopharmaceuticals.

The extraction of bioactive compounds from mushrooms from a variety of mushroom types aids in creating a sustainable supply of mushroom bioactive compounds for applications, for example, pharmaceuticals and nutraceuticals. Mushroom extracts containing polysaccharides, terpenoids, polyphenols, and other bioactive compounds are utilized in pharmaceutical formulations and nutraceutical supplements for their immunomodulatory, anti-inflammatory, antioxidant, and anticancer properties.

Other applications are in cosmetics and skincare. Mushroom-derived extracts and compounds are incorporated into cosmetics, skincare products, and cosmeceuticals for their moisturizing, anti-aging, and skin-brightening effects. Beta-glucans, polysaccharides, and phenolic compounds from mushrooms are prized for their hydrating, collagen-stimulating, and antioxidant properties.

Yet another application of the extracted mushroom bioactive compounds is in functional foods and beverages. Mushroom extracts are added to functional foods, beverages, and dietary supplements to enhance their nutritional profile and health benefits. Mushroom-derived polysaccharides, beta-glucans, and ergosterol are sought after for their cholesterol-lowering, immune-boosting, and prebiotic effects.

The extraction of bioactive compounds from mushrooms is a multifaceted process that involves a combination of methods, techniques, and applications. Extraction of the diverse chemical composition of mushrooms provides the full potential of mushroom-derived components for pharmaceutical, nutraceutical, cosmetic, and biotechnological applications. Mushrooms, with their long history of use in traditional medicine systems, are perceived as natural and holistic remedies for health conditions.

Advances in scientific research have elucidated the pharmacological properties and therapeutic potential of mushroom-derived bioactive compounds, including polysaccharides, beta-glucans, terpenoids, and polyphenols. Clinical studies and preclinical research support the efficacy of mushroom extracts in modulating immune function, reducing inflammation, and combating oxidative stress, paving the way for the development of mushroom-based medical products.

The global burden of chronic diseases such as cancer, diabetes, cardiovascular disorders, and immune-related conditions continues to rise, driving demand for complementary and alternative therapies. Mushroom-derived medical products offer potential solutions for managing symptoms, improving quality of life, and supporting conventional treatment regimens for chronic diseases.

There is a growing emphasis on preventive healthcare and wellness promotion, with consumers seeking proactive measures to maintain health and vitality. Mushroom-derived medical products, known for their immune-modulating, antioxidant, and anti-inflammatory properties, are positioned as preventive supplements for bolstering immune function, enhancing resilience, and promoting overall well-being.

The benefits of these bioreactors include increased efficiency and productivity. The bioreactors provide precise control of growth conditions, nutrient supply, and metabolic pathways, leading to enhanced yields, faster production rates, and improved process efficiency. Bioreactors facilitate quality control and process monitoring with real-time monitoring and analysis of process parameters, allowing early detection of deviations, optimization of conditions, and ensuring consistent product quality and purity. The bioreactors are highly scalable, allowing a seamless transition from laboratory-scale research to industrial-scale production. The bioreactors can be adapted to accommodate varying volumes, configurations, and process requirements, providing flexibility in process development and optimization.

The bioreactors are beneficial by providing reduced environmental impact. The bioreactors offer sustainable solutions for resource utilization, waste minimization, and eco-friendly production processes, contributing to environmental conservation and reducing the ecological footprint of industrial activities. These bioreactors advance personalized medicine solutions to address pressing global challenges in healthcare, energy, and sustainability in cultivating biological organisms.

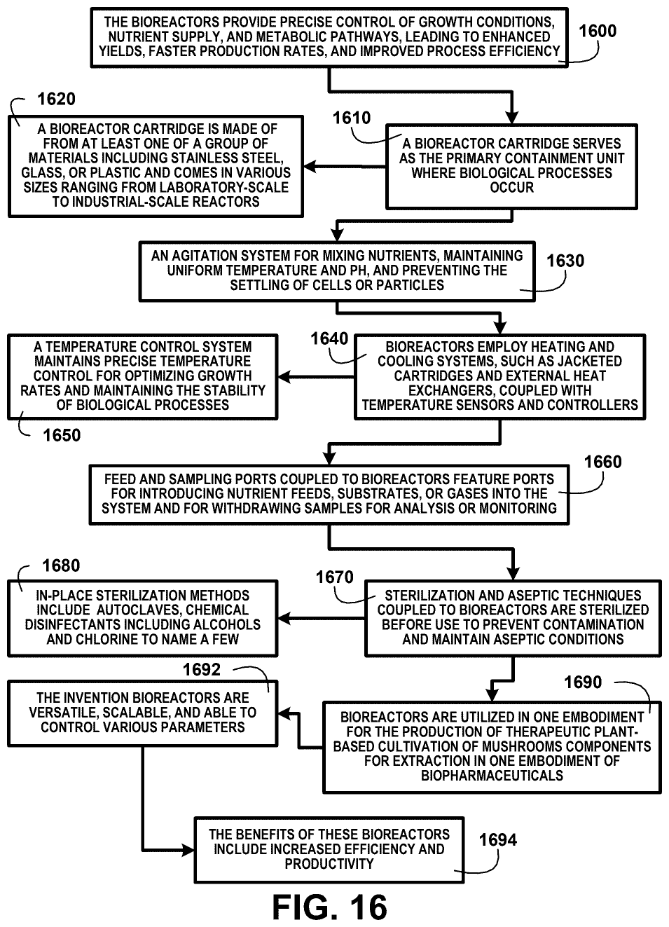

FIG. 16 shows a block diagram of an overview of bioreactor control of growth conditions of one embodiment. FIG. 16 shows the bioreactors provide precise control of growth conditions, nutrient supply, and metabolic pathways, leading to enhanced yields, faster production rates, and improved process efficiency 1600. A bioreactor cartridge serves as the primary containment unit where biological processes occur 1610. The bioreactor cartridge is made of at least one of a group of materials including stainless steel, glass, or plastic, and comes in sizes ranging from laboratory-scale to industrial-scale reactors 1620.

An agitation system for mixing nutrients, maintaining uniform temperature and pH, and preventing the settling of cells or particles 1630. Bioreactors employ heating and cooling systems, such as jacketed cartridges and external heat exchangers, coupled with temperature sensors and controllers 1640. A temperature control system maintains precise temperature control for optimizing growth rates and maintaining the stability of biological processes 1650. Feed and sampling ports coupled to bioreactors feature ports for introducing nutrient feeds, substrates, or gases into the system and for withdrawing samples for analysis or monitoring 1660.

Sterilization and aseptic techniques coupled to bioreactors are sterilized before use to prevent contamination and maintain aseptic conditions 1670. In-place sterilization methods include autoclaves, and chemical disinfectants including alcohols and chlorine to name a few 1680. Bioreactors are utilized, in one embodiment, for the production of therapeutic plant-based cultivation of mushroom components for extraction in one embodiment of biopharmaceuticals 1690. The invention bioreactors are versatile, scalable, and able to control parameters 1692. The benefits of these bioreactors include increased efficiency and productivity 1694 of one embodiment.

This invention integrates a plurality of sensors to monitor and actuate operating adjustments in, for example, temperature settings to maintain the bioreactor temperatures within predetermined ranges in one embodiment a range of 25° C. The plurality of sensors and actuators to automatically maintain operating protocols are wirelessly coupled to processors, databases, and communication devices.

The processors perform the analytics using proprietary coding and algorithms to determine mushroom culture status. Artificial intelligence and machine learning analyze photographic videos and images throughout the culturing to establish predictable growth patterns and rates of growth to compare current conditions with database-stored growth patterns and rates of growth.

The communication devices receive the sensor measurement and sensing data for transmission to the databases. The communication devices using Wi-Fi, internet, satellite, and other modes of bidirectional transmission further transmit wirelessly the processor operational monitoring results to the actuators to perform corrective action to bring the controls with the predetermined ranges of operation. The communication devices further transmit wirelessly alerts to the users to any out-of-performance protocols to initiate inspections of the equipment being monitored for any corrective maintenance or replacement of the equipment in a timely manner to prevent damage to the culturing operations and extraction operations biomasses.

The invention includes a digital app installed on user mobile devices and a remote computer 110 of FIG. 1 to receive sensing data and status reporting. The computer 110 of FIG. 1, processors, databases, and other appropriate devices may be coupled to a remote server. The remote server may generate reports of the production, quality status, and any regulatory compliance reports for proper certifications of the bioreactor cultivation and extraction results. The periodic reporting may be prepared for submission to appropriate local, state, and federal government agencies.

The sensors include sensing of pathogens, bacteria, and other organic constituents. Temperature, humidity, oxygenation levels, lighting levels, and other factors contribute to the detailed status of the cultivation and extraction processes. The remote server may also generate periodic maintenance reviews, predicted harvesting schedules, and other events related to the production of the bioactive compound supply status.

Process optimization is obtained by the bioreactor cultivation optimizing protocols for specific plant species and target compounds. These cultivation optimizing protocols also allow for fine-tuning growth conditions, nutrient formulations, elicitation strategies, and metabolic engineering approaches to maximize the yield, quality, and consistency of medicinal plant extracts.

The bioreactor cultivation optimizing protocols are needed for the growth patterns of mushrooms cultivated in bioreactors. Mushroom cultivation has undergone a significant transformation with the advent of bioreactors, offering precise control overgrowth conditions and enhancing productivity. Understanding the growth patterns of mushrooms within bioreactors is crucial for optimizing cultivation strategies, improving yields, and ensuring consistent quality. The growth patterns of mushrooms cultivated in bioreactors, generate the factors influencing their development, morphology, and metabolic activity.

Mushroom cultivation in bioreactors typically progresses through distinct growth phases, each characterized by specific morphological and metabolic changes. These phases include an inoculation phase that marks the introduction of mushroom spawn or mycelium into the bioreactor substrate. Mycelial growth begins as the fungal hyphae colonize the nutrient-rich substrate, establishing a network of interconnected filaments. During the spawn run phase, mycelial growth accelerates, spreading throughout the substrate and colonizing available nutrients. The mycelium undergoes rapid expansion, forming a dense and homogeneous network of hyphae.

Primordia formation signals the initiation of fruiting body development. Triggered by environmental cues such as temperature, humidity, and light exposure, the mycelium begins to differentiate into primordial structures, known as pins or knots, at specific locations within the substrate. The fruiting phase is characterized by the emergence and maturation of mushroom fruiting bodies from the primordia. Under optimal conditions of humidity, temperature, and air exchange, the fruiting bodies undergo rapid growth, development, and morphological differentiation, culminating in mature mushrooms ready for harvest.

Several factors influence the growth patterns and productivity of mushrooms cultivated in bioreactors, including the choice of substrate and nutrient composition profoundly impact mycelial growth, fruiting body formation, and yield. Substrates rich in carbon and nitrogen sources, such as sawdust, straw, or agricultural residues, provide essential nutrients for fungal metabolism and development. Environmental factors such as temperature, humidity, light, and air exchange play a critical role in regulating mushroom growth and development. Optimal conditions promote primordia initiation, fruiting body formation, and maturation.

Agitation and mixing within the bioreactor facilitate uniform distribution of nutrients, oxygenation of the substrate, and removal of metabolic byproducts. Proper agitation ensures optimal mycelial growth and prevents substrate compaction or anaerobic conditions. pH regulation is for maintaining the physiological balance of the mushroom mycelium and optimizing enzymatic activity. Monitoring and adjusting pH levels within the bioreactor substrate help create favorable conditions for mycelial growth and fruiting body development.

Optimizing mushroom cultivation in bioreactors requires a multifaceted approach encompassing substrate formulation, environmental control, and process optimization. Optimization strategies include tailoring substrate formulations to meet the nutritional requirements of specific mushroom species and optimizing pretreatment processes to enhance substrate accessibility and digestibility for fungal colonization.

Implementing precise control and monitoring systems for temperature, humidity, light, and air exchange to create optimal conditions for mycelial growth, primordia formation, and fruiting body development.

The invention provides bioreactors with appropriate agitation, mixing, aeration, and temperature control systems to promote uniform substrate colonization, prevent substrate stratification, and ensure consistent mushroom growth throughout the cultivation cycle.

Optimization includes supplementing bioreactor substrates with additional nutrients, growth promoters, or elicitors to enhance mycelial proliferation, induce primordia formation, and stimulate secondary metabolite production in mushrooms.

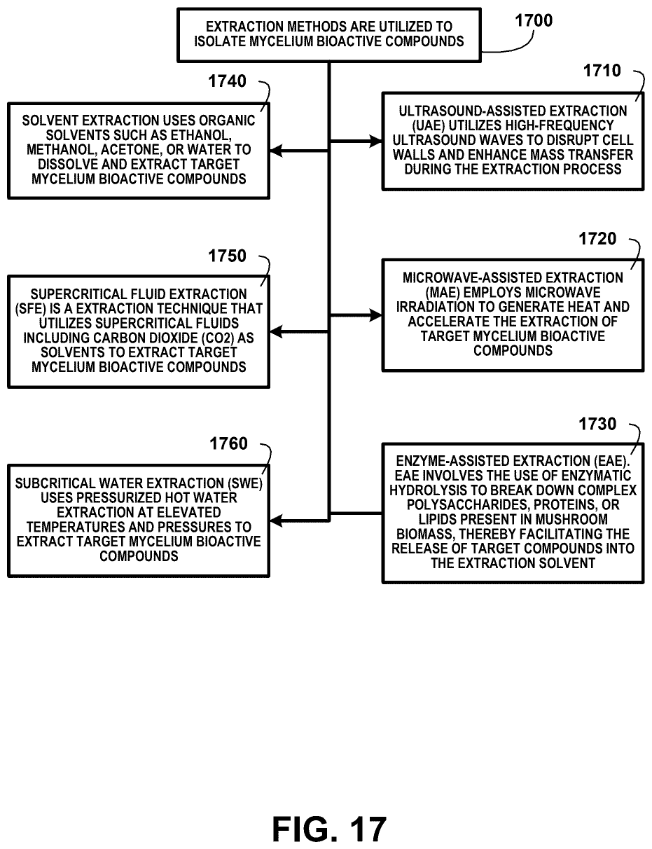

FIG. 17 shows a block diagram of an overview of the extraction of mycelium bioactive compounds of one embodiment. FIG. 17 shows extraction methods are utilized to isolate mycelium bioactive compounds 1700. Ultrasound-assisted extraction (UAE) utilizes high-frequency ultrasound waves to disrupt cell walls and enhance mass transfer during the extraction process 1710. Microwave-assisted extraction (MAE) employs microwave irradiation to generate heat and accelerate the extraction of target mycelium bioactive compounds 1720. Enzyme-assisted extraction (EAE) involves the use of enzymatic hydrolysis to break down complex polysaccharides, proteins, or lipids present in mushroom biomass, thereby facilitating the release of target compounds into the extraction solvent 1730.

Solvent extraction uses organic solvents such as ethanol, methanol, acetone, or water to dissolve and extract target mycelium bioactive compounds 1740. Supercritical fluid extraction (SFE) is an extraction technique that utilizes supercritical fluids including carbon dioxide (CO2) as solvents to extract target mycelium bioactive compounds 1750. Subcritical water extraction (SWE) uses pressurized hot water extraction at elevated temperatures and pressures to extract target mycelium bioactive compounds 1760 of one embodiment.

Another embodiment of extraction is subcritical water extraction (SWE). SWE, also known as pressurized hot water extraction or hydrothermal extraction, utilizes water at elevated temperatures and pressures to extract target compounds from mushroom biomass. SWE is effective for extracting polar and semi-polar compounds, such as polysaccharides and phenolic compounds, while minimizing the need for organic solvents and preserving the native structure and bioactivity of the extracted components. In addition to extraction methods, techniques are employed to enhance the efficiency, yield, and specificity of mushroom component extraction. Embodiments of extraction also include ultrasound-assisted extraction (UAE). UAE utilizes high-frequency ultrasound waves to disrupt cell walls and enhance mass transfer during the extraction process. Ultrasonic cavitation promotes the release of intracellular components from mushroom biomass, resulting in higher extraction yields and reduced extraction times. UAE is particularly effective for extracting polysaccharides, terpenoids, and phenolic compounds from mushrooms, offering advantages such as shorter extraction times, lower solvent consumption, and improved extraction efficiency.

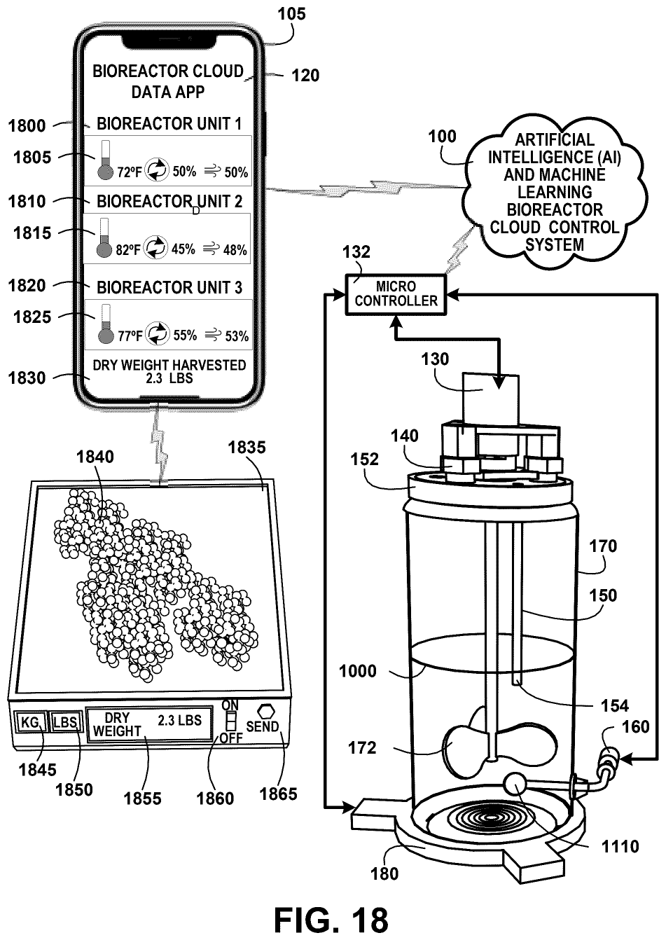

FIG. 18 shows for illustrative purposes only an example of the bioreactor cloud data app of one embodiment. FIG. 18 shows the artificial intelligence (AI) and machine learning bioreactor cloud control system 100 wirelessly coupled to the microcontroller 132 having the bioreactor cloud data app 120 and a user's mobile device 105 having a bioreactor cloud data app 120. The microcontroller 132 coupled to the artificial intelligence (AI) and machine learning bioreactor cloud control system 100 regulates the operations of at least a motor driver 130 coupled to magnetic motor support 140 of the lid 152, the agitator 172 coupled to the motor driver 130, the heating element base 180, the thermistor 154 inserted into the thermistor tube to measure the temperature of the substrate, and the oxygenation air pump 160 of FIG. 1 supplying airflow to the air stone 1110. The substrate level 1000 is shown in the bioreactor cartridge 170.

The user's mobile device 105 bioreactor cloud data app 120 displays the bioreactor units the user has registered, each with a unique serial number. Displayed is bioreactor unit 1-1800 showing unit 1 settings temp 72° F., rpm 50%, and oxygenation 50% 1805. Also shown are bioreactor unit 2 1810-unit 2 settings temp 82° F., rpm 45%, and oxygenation 48% 1815, and bioreactor unit 3 1820-unit 3 settings temp 77° F., rpm 55%, and oxygenation 53% 1825.

The user has placed the dried harvested mycelium on a user digital scale 1835 and transmitted the dry weight harvested 2.3 lbs. 1830 feedback to the bioreactor cloud data app 120. The feedback to the bioreactor cloud data app 120 transmits the user feedback to the artificial intelligence (AI) and machine learning bioreactor cloud control system 100. The harvested weight is analyzed by artificial intelligence (AI) and machine learning bioreactor cloud control system 100 with comparisons of other harvested outcomes and the operating settings during cultivation to determine settings that produce the optimum harvests. This analysis is used in making recommendations on settings to allow users to optimize the cultivation process.

The user digital scale 1835 weighs the dried harvested mycelium 1840 placed on the scale in kg 1845 or lbs. 1850 selected by the user. The user digital scale 1835 includes an on/off switch 1860 and displays the dry weight of 2.3 lbs. 1855 which the user can select to send 1865 of one embodiment.

The bioreactor system cultivates the mycelium root structures into a natural product. This product has an exact, consistent potency containing all the natural compounds found in these fungi. Typically, it takes 6-8 weeks to fully grow mushrooms, and using the bioreactor system the production of the active ingredients is only 7-10 days.

Traditional mushroom cultivation requires extensive infrastructure and costly lab equipment, posing significant barriers to small-scale producers. The bioreactor system reduces costs, simplifies the process, and ensures safe, high-quality production, making it accessible to a broader audience.

Naturally occurring bioactive compounds found in certain mushrooms have shown significant promise in clinical studies for treating mental health conditions such as depression, anxiety, PTSD, and addiction. Growing mycelium is an integral part of mushroom cultivation. It is most often grown by cloning it from one growth medium to another. Mushroom mycelium can also be grown from spores.

Mushroom-producing fungi are, by volume and mass, mostly composed of a filamentous mycelium. Mycelium looks like a web-like branching network of white fungal strands. Mycelium in nature can be found under leaf litter, rotting logs, and even sometimes in the soil. Only once matured will this mycelium begin producing mushrooms. Mycelium grows within substrates that act as their home and food source. Substrates are the natural materials that fungi inhabit. Common substrates are wood, straw, grain, cardboard, and organic waste. Mycelium grows from spores that are the reproductive cells of the mushroom. For cultivation, the spores are germinated. Cultivation of mycelium via spores is an advanced technique. This usually requires lab equipment and skill. For beginners, it is much better to start with a clean and healthy mycelium culture.

Mushroom spawn is mycelium, usually grown on grain and used for inoculation. Inoculation is the process of introducing mycelium into a new growth medium. High-quality mushroom spawns should grow vigorously and be free of any contaminants. Cultivation equipment is well-cleaned and rubbed down with 70% alcohol. Pasteurization is a process in which you remove antagonistic microorganisms from your substrate. There are many ways to pasteurize your substrate. The easiest is to submerge your substrate in water for 1 hour at 160° F. Alternatively, submerge your substrate in 0.2% activated lime water for 12 hours. After pasteurization, place your substrate to drain and cool down. If producing biomaterials, inoculate your mushrooms within a specialized mold. Make sure it has air exchange and proper growing conditions.

Incubate growing mushroom mycelium in liquid culture. Incubation promotes Mycelium growth in a liquid medium. The liquid medium in the nutrient container is supplemented with nutrients to provide proper growth ingredients to produce the best growth of the Mycelium. The incubator regulates an optimum temperature to stimulate growth of the spores. If the temperature is too low, the growth rate slows down. Low temperatures can lead to slower colonization of the substrate and reduced biomass accumulation The growth ingredients added to a sugar-water solution inside a nutrient container with a lid. The liquid solution within the nutrient container provides the proper mixture of nutrients for the specific Mycelium spore species. The lid of the nutrient container has a “self-healing injection port” to inoculate the liquid culture with best growth spores into new growth mediums with a syringe.

Temperature control modulators are configured to maintain the temperature settings stored in the bioreactor cloud platform. The temperature settings are based on the best growth results for each mycelium spore strain. The bioreactor cloud platform will automatically enter the temperature settings into the nutrient container and bioreactors upon start of a growth cycle. Optimum proper growth conditions produce higher yields of the best growth spores and increase harvests of the mycelium products.

Temperature plays a role in the cultivation of mycelium in a bioreactor. Mycelium has an optimal temperature range for growth. For many species, this range is between 20° C. and 30° C. (68° F. to 86° F.), though it can vary depending on the specific fungal strain. Within this range, the mycelium grows most efficiently, with optimal rates of nutrient uptake and metabolic activity. In a temperature within the optimal range, the mycelium grows rapidly. However, if the temperature is too low, the growth rate slows down. Low temperatures can lead to slower colonization of the substrate and reduced biomass accumulation.

Elevated temperatures can increase metabolic activity but can also stress the mycelium. If temperatures exceed the optimal range, it can lead to reduced growth rates, nutrient depletion, and even death of the mycelium. Prolonged exposure to elevated temperatures may also increase the risk of contamination. Low temperatures can slow down or halt growth. Prolonged exposure to low temperatures may lead to poor colonization and reduced yields. In some cases, extremely low temperatures might even cause damage to the mycelial structure.

Temperature affects not only growth but also the productivity and yield of the mycelium. Maintaining an optimal temperature can lead to higher yields and better quality of the fungal product. For example, in industrial applications, temperature control is crucial for maximizing mushroom production or the yield of mycelium-based products. Temperature influences metabolic processes within the mycelium, including enzyme activity and nutrient metabolism. Proper temperature regulation ensures that these processes occur efficiently, supporting healthy mycelial growth and development. Temperature also plays a role in controlling microbial contamination. Maintaining a stable and appropriate temperature can help minimize the risk of contamination by unwanted microorganisms. Controlling temperature in a bioreactor is for optimizing mycelium cultivation. Maintaining temperatures within the species-specific optimal range ensures efficient growth, productivity, and quality of the mycelial biomass.

Agitation of the growing medium in mycelium cultivation, in a bioreactor with a liquid culture system, plays a role in improving the growth and development of spores. Agitation helps to ensure an even distribution of oxygen throughout the growing medium. Mycelium, the vegetative part of fungi, requires a constant supply of oxygen to support aerobic respiration and overall growth. Agitation prevents oxygen depletion and promotes efficient gas exchange. Agitation helps to evenly distribute nutrients throughout the medium. This ensures that all parts of the growing medium receive an adequate supply of nutrients, for consistent and healthy mycelial growth.

Agitation can aid in maintaining uniform temperature distribution within the bioreactor. It helps to prevent localized temperature gradients that could negatively impact mycelial growth and development. In liquid cultures, agitation prevents the settling of mycelium or spores at the bottom of the container. This is particularly important in liquid fermentation systems where uniform growth throughout the medium is desired. Agitation ensures a homogeneous suspension of spores or mycelium in the medium. This is important for maintaining consistency in growth rates and ensuring that the mycelium colonizes the medium evenly.

By improving oxygen and nutrient availability, agitation can lead to faster growth rates of the mycelium. This can be beneficial in both research and industrial applications where rapid mycelial expansion is desired. Agitation can help to reduce the risk of contamination by preventing the establishment of localized conditions where contaminants might thrive. However, it is important to balance agitation with other factors to minimize the risk of mechanical damage to the mycelium or excessive shear forces that might stress it. Overall, agitation is a factor in optimizing the growth environment for mushroom spores in liquid cultures or bioreactors. It helps to create a more uniform and favorable growth environment, supporting the efficient and healthy development of the mycelium.

Oxygenation is used in cultivating mycelium in a bioreactor. Mycelium, being an aerobic organism, relies on oxygen for physiological and metabolic processes. Mycelium primarily uses aerobic respiration to generate energy. Oxygen is required for the complete breakdown of substrates, leading to the production of ATP (adenosine triphosphate), which is essential for growth and metabolism. Inadequate oxygenation can lead to a shift from aerobic to anaerobic respiration, which is less efficient and can result in the accumulation of toxic by-products.

Adequate oxygen levels support optimal growth rates and biomass production. In a bioreactor, sufficient oxygenation ensures that the mycelium grows efficiently, resulting in higher yields and better quality of the fungal product. Oxygenation affects nutrient uptake and utilization. Proper oxygen levels enhance the efficiency of nutrient metabolism, which is crucial for maintaining healthy mycelial growth and development.

In large-scale or high-density cultures, oxygen demand can exceed the supply if not effectively managed. Oxygen limitation can lead to reduced growth rates, slower colonization, and poor overall performance. Effective aeration strategies are needed to ensure that oxygen is adequately supplied throughout the culture. Inadequate oxygenation can lead to the production of unwanted metabolic by-products, such as ethanol and other fermentation products. These by-products can inhibit growth and affect the overall health of the mycelium.

In a bioreactor, effective oxygenation helps in maintaining a uniform environment. This includes the even distribution of oxygen throughout the culture medium, preventing localized areas of low oxygen that can negatively impact growth. The design of the bioreactor, including agitation and aeration systems, is often tailored to meet the oxygen demands of the mycelium. Properly designed bioreactors ensure that oxygen is efficiently supplied and distributed, supporting optimal growth conditions.

In summary, oxygenation increases the successful cultivation of mycelium in a bioreactor. It directly impacts growth rates, productivity, nutrient utilization, and overall health of the mycelium. Effective oxygen management strategies are essential to maintain a favorable growth environment and achieve desired outcomes in mycelium cultivation.

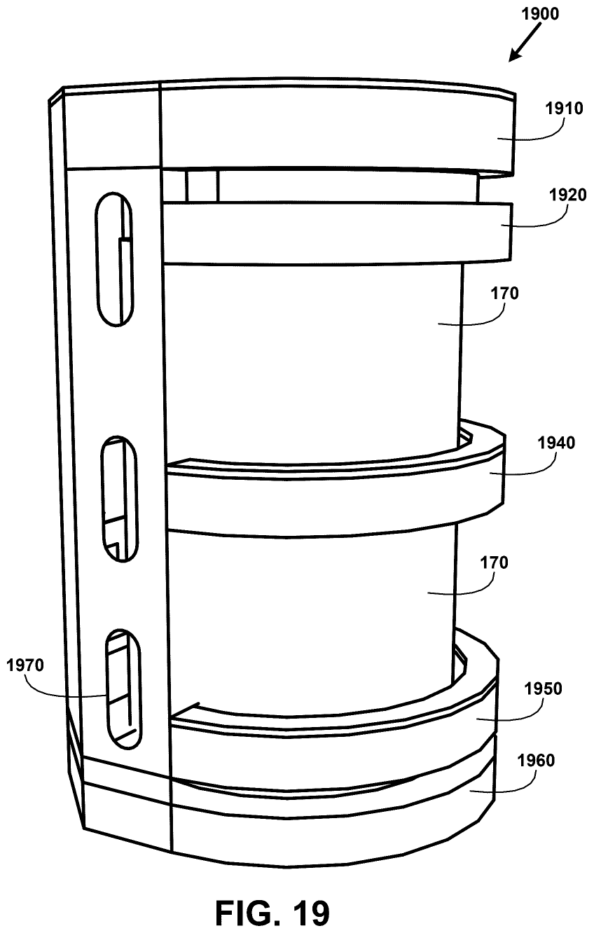

FIG. 19 shows for illustrative purposes only an example of a magnetic collar of one embodiment. FIG. 19 shows a bioreactor 1900 having a plurality of a magnetic collar 1910. The bioreactor cartridge 170 is held tightly in place with the plurality of a magnetic collar 1910 including magnetic collar 1910, magnetic collar 1920, magnetic collar 1940, magnetic collar 1950, and magnetic collar 1960. The bioreactor 1900 includes ventilation openings 1970 to provide airflow to control the temperature within the bioreactor of one embodiment.

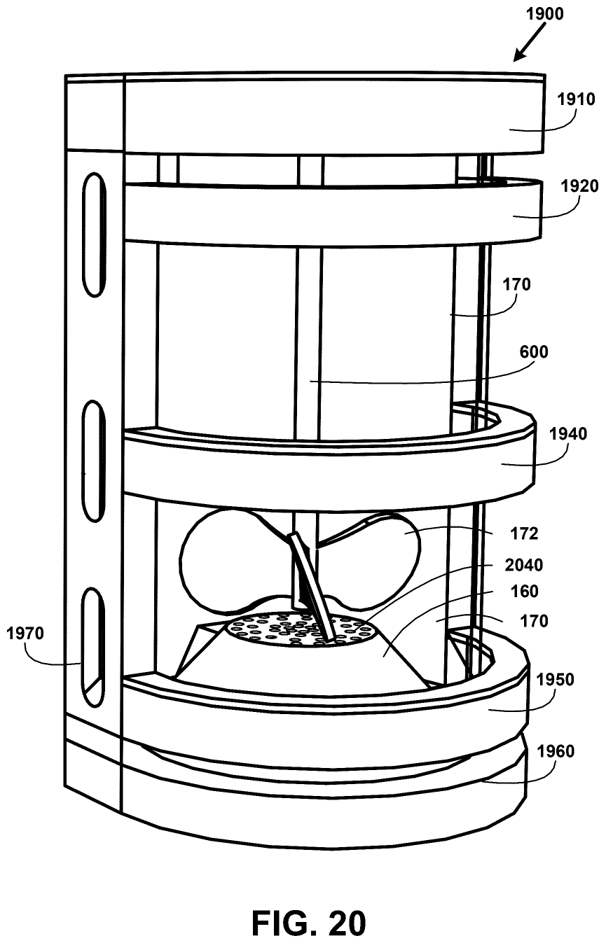

FIG. 20 shows for illustrative purposes only an example of an agitator rotor rod of one embodiment. FIG. 20 shows the bioreactor 1900 having magnetic collar 1910, magnetic collar 1920, magnetic collar 1940, magnetic collar 1950, and magnetic collar 1960 used to secure tightly the bioreactor cartridge 170. The bioreactor 1900 includes ventilation openings 1970 to provide airflow to control the temperature within the bioreactor. Also showing is the agitator rotor rod 600 coupled to the agitator 172. The oxygenation air pump 160 pumps oxygen through cartridge oxygenation airflow inlets 2040 to provide oxygen to the sterile substrate to promote the growth of the mushroom spores of one embodiment.

The bioreactor cartridge contains a sterile substrate into which mycelium spores are injected prior to placing into the bioreactor. The bioreactor includes a front pivoting section of the top section to open for the installation of a bioreactor cartridge. When placed in the bioreactor the motor is connected to the cartridge agitator rod. Upon closing the pivoting section magnetic collars are coupled to the bioreactor to hold the cartridge securely in place. Gears are engaged to operate the agitator motor. The control display screen is activated. The heating element is located at the bottom of the bioreactor to heat the sterile substrate and spores. The air filter and oxygenation aerator motor are connected to the microcontroller and air stone tube to begin injecting oxygen into the sterile substrate and spores. The bioreactor cartridge begins operations for rotating the agitator, oxygenation aerator motor, and heating the sterile substrate and spores.

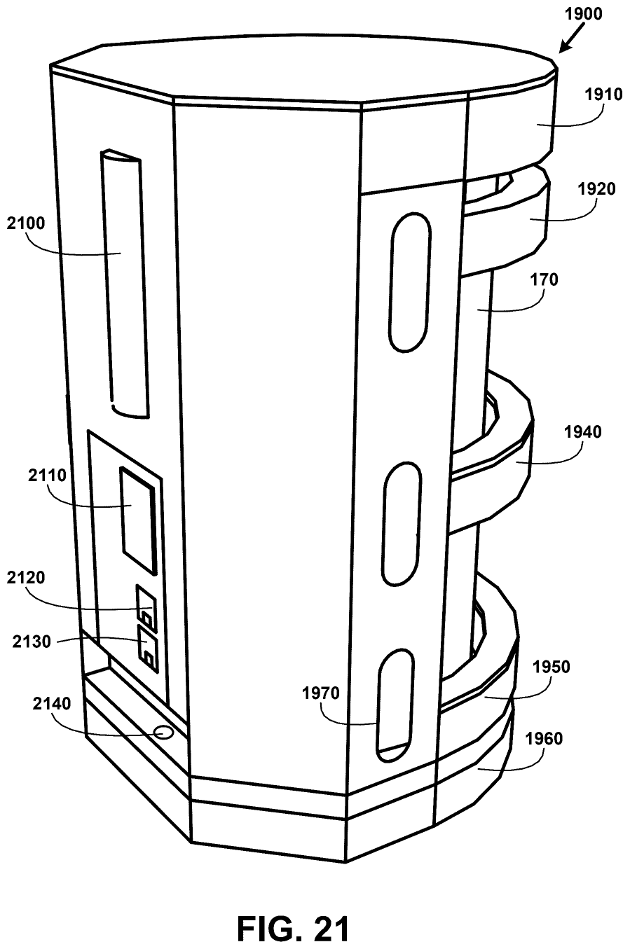

FIG. 21 shows for illustrative purposes only an example of a bioreactor cartridge controls of one embodiment. FIG. 21 shows the bioreactor 1900 having the magnetic collar 1910, magnetic collar 1920, magnetic collar 1940, magnetic collar 1950, and magnetic collar 1960 used to secure the bioreactor cartridge 170. The bioreactor 1900 includes ventilation openings 1970 to provide airflow to control the temperature within the bioreactor. Also showing is one embodiment of a bioreactor cartridge thermistor tube 2100 for insertion of a thermistor 154 of FIG. 1. FIG. 21 also shows a control display screen 2110 to display the temperature, oxygen level, and rpm of the agitator to allow the user to monitor the cultivation progress.

Also shown is an oxygen saturation gauge 2120 to display the saturation level of oxygen in the substrate for adjusting the flow rate of the oxygenation air pump 160 of FIG. 1. A nutrient sensor gauge 2130 displays sensor readings of nutrient levels in the substrate to determine supplemental nutrient amounts to be added to the substrate. A reset power button 2140 allows the user to reset the gauges to get accurate current readings.

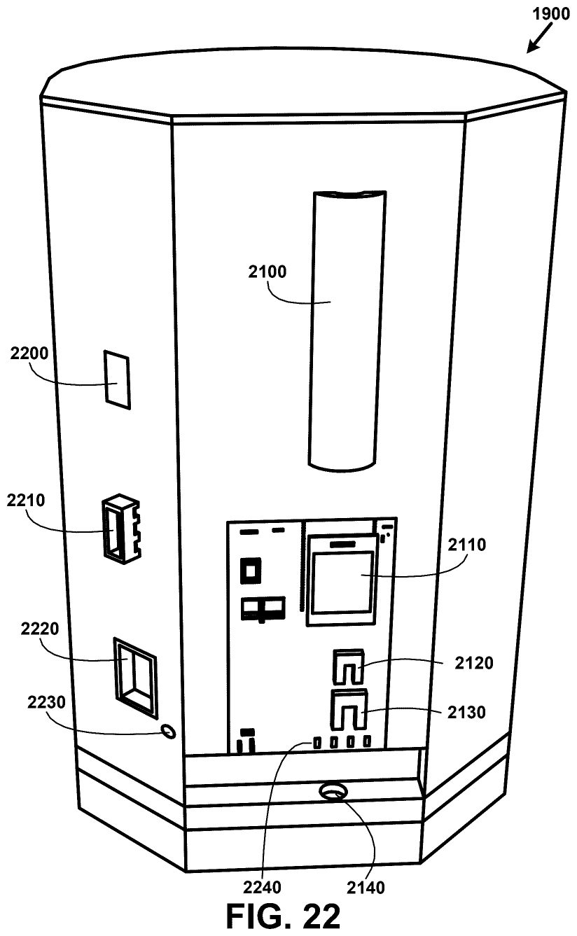

FIG. 22 shows for illustrative purposes only an example of the control display screen of one embodiment. FIG. 22 shows the bioreactor cartridge thermistor tube 2100 and the control display screen 2110. Also seen are the oxygen saturation gauge 2120, nutrient sensor gauge 2130, and reset power button 2140. Also showing are LED status lights 2240. A power light 2200 is lit when power is connected to the bioreactor 1900 and not lit when power is off. A USB connector 2210 allows the user to connect USB devices to, for example, record current readings for historical progress of the cultivation. A substrate sample outlet 2220 allows the user to get a test sample of the substrate for testing cultivation conditions that affect the growth of the mushroom spores 176 of FIG. 1. A sample button 2230 is used to turn on a suction pump to extract the substrate sample of one embodiment.

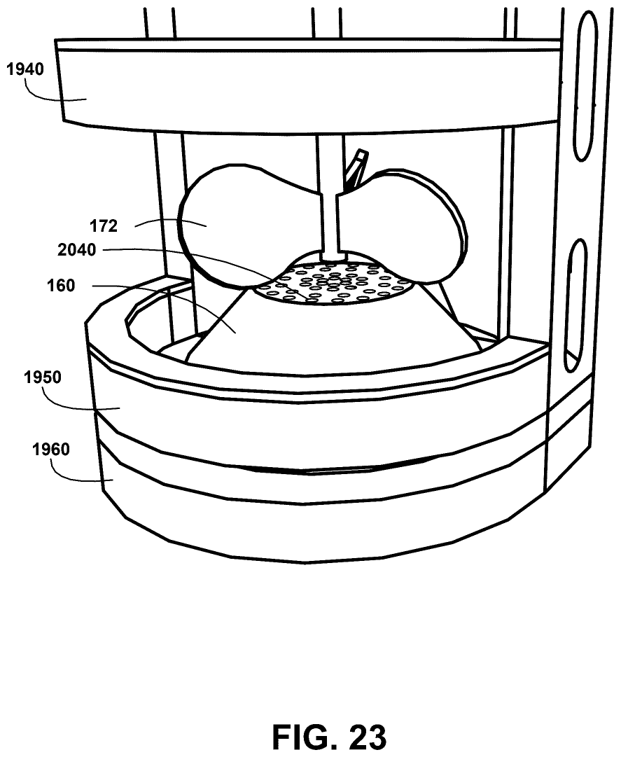

FIG. 23 shows for illustrative purposes only an example of oxygenation air flow vents of one embodiment. FIG. 23 shows the magnetic collar 1940, the magnetic collar 1950, and the magnetic collar 1960. Also showing is the agitator 172, oxygenation air pump 160, and oxygenation air flow vents 2040.

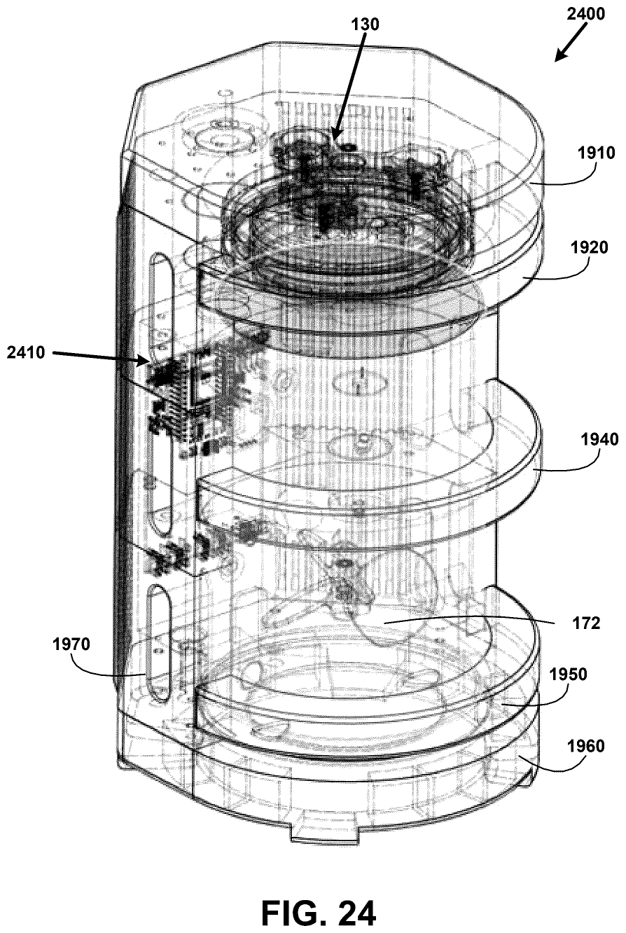

FIG. 24 shows for illustrative purposes only an example of a transparent view of the bioreactor of one embodiment. FIG. 24 shows a transparent view of the bioreactor 2400 with the bioreactor cartridge secured with the magnetic collar 1910, magnetic collar 1920, magnetic collar 1940, magnetic collar 1950, and magnetic collar 1960.

The ventilation openings 1970 provide airflow to control the temperature within the bioreactor 1900. The control panel 2410 is seen from the back inside view and includes the control components. The motor driver 130 is seen on top and is used to rotate the agitator 172 at predetermined rpm settings that are adjusted using artificial intelligence to regulate the settings of one embodiment.

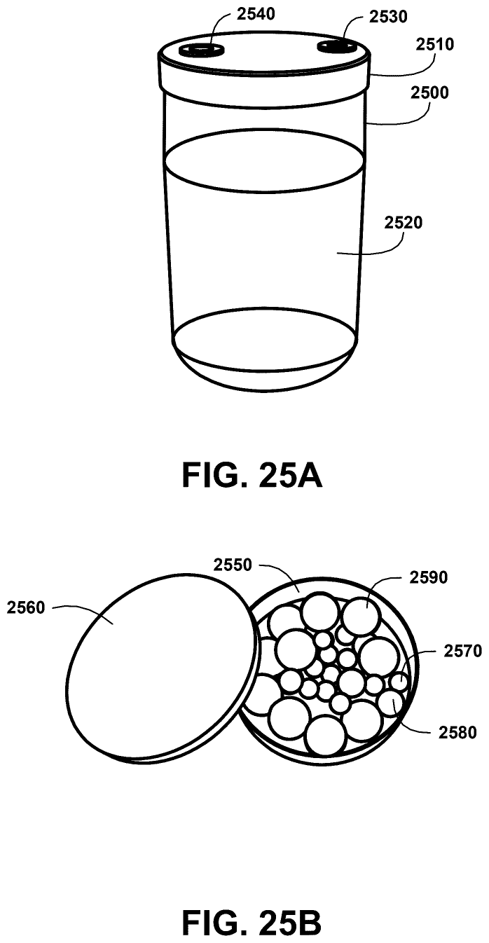

FIG. 25A shows for illustrative purposes only an example of mycelium liquid culture-nutrients in a liquid solution of one embodiment. FIG. 25A shows a nutrient liquid container 2500 with a cover 2510. Inside the container is a sterile liquid substrate 2520, to which nutrients are added through a dry nutrient fill port 2530. This mixture provides essential growth ingredients for cultivating spores. Once prepared, the spores are injected into the nutrient solution, and the best growth spores identified based on growth are added to foster growth.

The culture continues to expand in the liquid, ensuring an adequate supply of spores for cartridge distribution. A cartridge is the growth module that is coupled to the bioreactor to monitor the growth of the mycelium inoculated into the nutrient solution held in the cartridge. The bioreactor receives settings for the control modules of the bioreactor that are coupled to the agitator, oxygenation aerator motor, and heating element of the cartridge. The agitator rpm is adjusted during the growth cycle to prevent the spores from clumping together as more growth occurs, provides spacing between spores for access to oxygen and nutrients. The oxygenation aerator motor passing air through an air filter to provide oxygen to the spores to maintain growth of the spores. The heating element is regulated to provide heat within the optimum range of the spore strain. The settings are adjusted by the cloud platform as the growth cycle progresses. The cartridge includes an injection port to add supplemental nutrients to keep the nutrient levels at a level to maintain adequate supplies to the spores as the numbers increase with the growth.

The cartridges are sent to users with the sterile liquid substrate within the cartridge. The cultured spores are sent separately in a syringe to inject into the cartridge to begin the growth cycle.

The supply of spores for different strains are initially cultivated using an incubator to regulate the temperature with the optimum range of temperatures for each strain held in a dish. After 7 to 14 days the spore growth is evaluated, and the best growth spores are segregated from poorer growth spores for inoculation into a nutrient container. The nutrient container holds a liquid solution to continue the growth of the best growth spores. The growth of the spores in the nutrient container builds a larger supply of the best growth spores to extract with a syringe to feed the spores into the cartridge.

A liquid nutrient extraction port 2540 allows sterile extraction of the nutrient solution, which can be transferred into cartridges without living organisms, awaiting spore inoculation. The nutrient solution contains components tailored to support mycelium strains.

FIG. 25B shows for illustrative purposes only an example of plating spores to culture of one embodiment. FIG. 25B shows a mycelium culture dish 2550 and a dish cover 2560. Spores are placed in the dish with nutrients and cultured for 7 to 14 days. Afterward, the spores are genetically analyzed to classify them into three categories: poor growth spores 2570, medium growth spores 2580, and best growth spores 2590. The best growth spores 2590 are selected, and poor or medium spores are removed. The best spores continue to grow and multiply, in one embodiment moved to larger cartridges to ensure an adequate quantity. This process ensures a strong culture for inoculation into nutrient solutions.

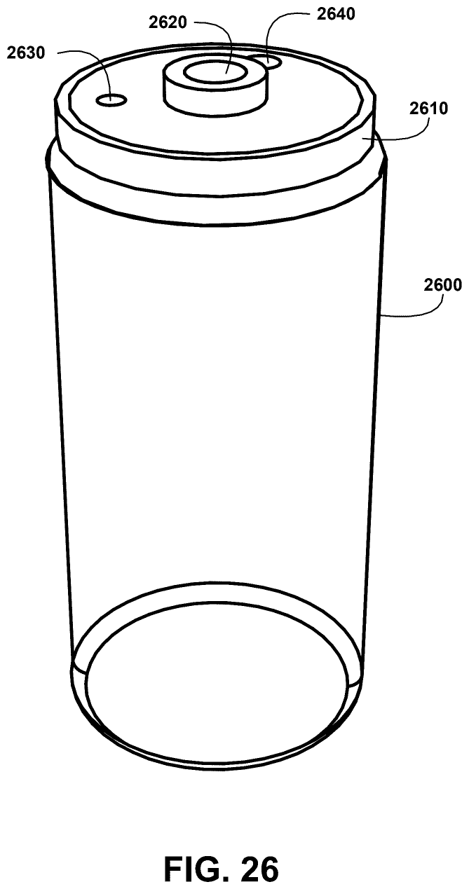

FIG. 26 shows for illustrative purposes only an example of refillable growing medium cartridge of one embodiment. FIG. 26 shows a refillable growing medium cartridge 2600 with a cover 2610 that contains an injection sterile port 2620, a nutrient injection port 2630, and a pressure relief valve 2640. The pressure relief valve helps manage excess pressure created when heating spores in the nutrient solution. The sterile port allows for spore injection into a bioreactor container cartridge referred to as part A, where spores are introduced using a 10 cc syringe after the nutrient solution has been added. The cartridge part B contains liquid nutrients such as dextrose, sugars, and nitrogen that support mycelium growth. The best growth spores are injected into this nutrient-rich solution.

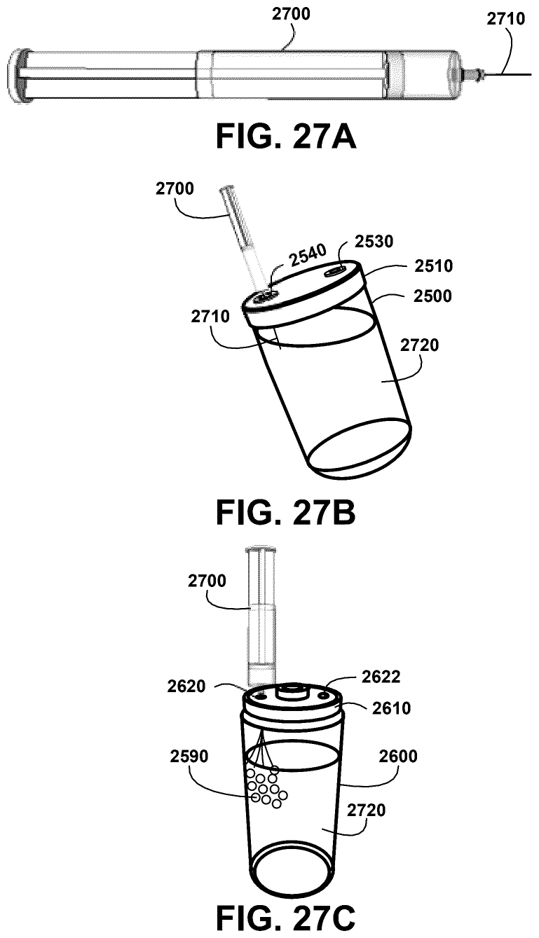

FIG. 27A shows for illustrative purposes only an example of a syringe of one embodiment. FIG. 27A shows illustrates a 10 cc syringe 2700 with an attached needle 2710. The syringe is filled using a sterile process and shipped in sterile packaging. It is used for injecting best growth spores 2590 of FIG. 25B into the liquid growth nutrients 2720 of FIG. 27B in the cartridge, maintaining the sterile environment.

FIG. 27B shows for illustrative purposes only an example of syringe extracting liquid nutrient of one embodiment. FIG. 27B shows the syringe 2700 and needle 2710 extracting liquid growth nutrients 2720 through the extraction port 2540 on the cover 2510, which are mixed with cultured best growth spores 2590 of FIG. 25B from the nutrient liquid container 2500. Nutrients are added through the dry nutrient fill port 2530, and the liquid nutrient extraction port 2540 is used to extract nutrient solution without living organisms, or with best growth spores, depending on the process.

FIG. 27C shows for illustrative purposes only an example of syringe injecting best growth spores of one embodiment. FIG. 27C shows the syringe 2700 injecting best growth spores 2590 into the liquid growth nutrients 2720 contained in the bioreactor cartridge 2600. The container holds a liquid substrate with liquid growth nutrients 2720. The cover 2610 of the container features both the dry nutrient fill port 2622 and the sterile port 2620 for injecting the nutrients and spores.

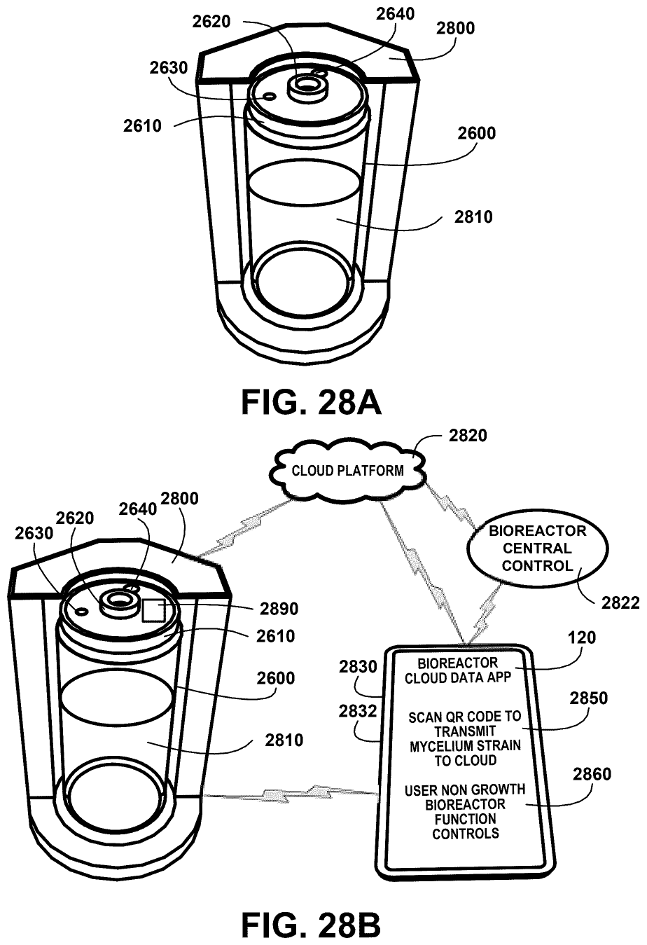

FIG. 28A shows for illustrative purposes only an example of refillable growing medium cartridge placed in bioreactor of one embodiment. FIG. 28A shows the refillable growing medium cartridge 2600 placed inside a bioreactor 2800. The cartridge cover 2610 includes a sterile port 2620 for spore injection into the liquid nutrient 2810. The nutrient injection port 2630 is used to add additional nutrients as needed during the cultivation process. Also present is a pressure relief valve 2640 to prevent pressure build-up.

FIG. 28B shows for illustrative purposes only an example of a bioreactor app of one embodiment. FIG. 28B shows the refillable growing medium cartridge 2600, equipped with an aeration system to prevent spore clumping. Part B of the system is recyclable and can be returned for sterilization and refilling. The cartridge cover 2610 includes the sterile port 2620, nutrient injection port 2630, and a pressure relief valve 2640.

The bioreactor app includes user selection from the dashboard of sections that provide user guidance in a grow or a new recipe. The sections include APEX 1, WILL1, COSMIC GAZE, SUPERNOVA, and BLACK STAR. APEX 1 allows the user to review monitoring displays of temperature, rotation speed, and air agitation. WILL1 provides the user with a manual or new recipe selection. The manual selection allows the user to configure the set points of the mushroom accelerator and change the set points when the user sees fit. The new recipe selection allows the recipe to automatically schedule set point changes to the mushroom accelerator and allow the changes to take place. The COSMIC GAZE section displays the current actual temperature with a degree reading, rotation speed with a percentage of a 100% rate, and air agitation with a percentage of a 100% rate. The SUPERNOVA section displays the current actual temperature with a degree reading, rotation speed with a percentage of a 100% rate, and air agitation with a percentage of a 100% rate and a graphical chart that depicts the changes in set point responses.

The SUPERNOVA section includes a subsection ANALYTICS that displays three factors including temperature, agitator motor speed, and oxygenation air speed. The user may select a grow period of days and compare the set point settings to the actual average of the three factors over the number of calendar days chosen by the user. This information allows the user to make adjustments, if necessary, in set points to more closely follow a recipe. The display further includes a timeline when set points have been made to further inform the user of the effectiveness of the particular set point changes. The user may also change the set points in the subsection ANALYTICS with drop down features to select a set point and the date and time the change will take effect and change the color of the chart display.

The SUPERNOVA section includes a subsection of ATTRIBUTES. ATTRIBUTES includes GROWS to record current cultivation facts including Micropearl Type, Growth Start, Growth End, Started By, And Grow ID. Displayed adjacently are the same facts from previous Historical cultivations with, for example, dates and times, name of type of mycelium, harvest results and other corresponding information. Settings may be reloaded and rebooted.

The bioreactor app further includes a store from which to order different mycelium species including for example Enoki, Lion's Mane, and Shiitake. The order form lists the prices and a purchase cart.

A user's smartphone 2830 has installed a bioreactor cloud data app 120 to allow the user to interact with the refillable growing medium cartridge 2600, the cloud platform 2820, and bioreactor central control 2822. The user's smartphone 2830 is used to scan QR code to transmit mycelium strain to cloud 2850. The user may operate with the bioreactor cloud data app 120 user non growth bioreactor function controls 2860.

A QR code 2890 is attached to the cartridge cover, containing details about the mycelium strain. When scanned by the user's smartphone camera 2832, the QR code transmits information to a cloud platform 2820. The cloud platform 2820 sends control settings for the bioreactor, such as agitation rate, temperature, and oxygen flow, optimizing the environment for mycelium growth. The bioreactor cloud data app 120 also allows the user to control non-growth functions, for example, LED light color. The bioreactor central control 2822 generates the QR code for identifying cultured mycelium and sends batch progress updates to the user, making adjustments to the bioreactor as the culture develops.

In one embodiment a portable and compact bioreactor for controlled cultivation of mycelium spores includes a bioreactor cartridge with an injection port, wherein the bioreactor cartridge is removably coupled to the bioreactor and contains a sterile substrate comprising organic materials as a nutrient, and wherein the injection port is a sealed, one-way sterile inlet configured to introduce the mycelium spores and predetermined nutrients into the sterile substrate while preventing contamination.

Further including components comprising a pressure relief valve coupled to the bioreactor configured to prevent pressure build-up from heating the sterile substrate; a bioreactor microcontroller coupled to the bioreactor cartridge configured to monitor heat, oxygen, and nutrient levels; a heating element coupled to the bioreactor microcontroller configured to heat the sterile substrate within the bioreactor cartridge to a predetermined temperature; an air pump coupled to the bioreactor cartridge and to the bioreactor microcontroller configured to supply oxygenation of the mycelium spores; an agitator located within the bioreactor cartridge and coupled to a motor driver coupled to the bioreactor microcontroller and configured to rotate at a predetermined speed to distribute heat, oxygen, and nutrients among the mycelium spores with predetermined parameters; a cloud control system coupled to the bioreactor microcontroller configured to analyze empirical data related to cultivation of the mycelium spores to determine the predetermined speed, the predetermined parameters and the predetermined nutrients to produce specific harvest results and to make future automatic adjustments to the predetermined speed, the predetermined parameters and the predetermined nutrients based on the specific harvest results; and a bioreactor cloud data app coupled to a mobile device of the user configured to remotely monitor settings of the bioreactor, to allow the user to adjust the settings and to receive recommended settings based on the analysis of the cloud control system.

In yet another embodiment the portable and compact bioreactor for controlled cultivation of mycelium spores is further comprising an artificial intelligence (AI) and machine learning bioreactor cloud control system wirelessly coupled to a bioreactor cloud data app coupled to a user's mobile device configured to remotely monitor settings of the bioreactor, to allow the user to remotely adjust the settings and to receive recommended settings based on the analysis of the cloud control system. Wherein the bioreactor cloud data app coupled to a user's mobile device is further configured to allow the user to self-report harvest results to the artificial intelligence (AI) and machine learning bioreactor cloud control system. The bioreactor microcontroller bioreactor cloud data app is configured to receive parameter adjustment settings to automatically regulate parameter settings and optimize cultivation results in the bioreactor cartridge based on the analytical input data from the artificial intelligence (AI) and machine learning bioreactor cloud control system including user self-reported harvest results. Wherein the empirical data is transmitted automatically to the artificial intelligence (AI) and machine learning bioreactor cloud control system from each bioreactor microcontroller, user mobile device via the bioreactor cloud data app. Further comprising the artificial intelligence (AI) and machine learning bioreactor cloud control system is further configured to compare past harvest results and past parameter settings to current harvest results and cultivation settings to determine optimal settings during cultivation that produce optimized harvest results to base recommended adjustments. Wherein the bioreactor includes a front pivoting section of a top cover section to pivot open for the installation of a bioreactor cartridge and make connections to the microcontroller, air pump oxygenation system, agitator drive and positioned above the heating element.

The foregoing has described the principles, embodiments, and modes of operation of the present invention. However, the invention should not be construed as being limited to the particular embodiments discussed. The above-described embodiments should be regarded as illustrative rather than restrictive, and it should be appreciated that variations may be made in those embodiments by workers skilled in the art without departing from the scope of the present invention as defined by the following claims.

What is claimed is:

1. A portable and compact bioreactor for controlled cultivation of mycelium spores, comprising:
a bioreactor cartridge with an injection port, wherein the bioreactor cartridge is removably coupled to the bioreactor and contains a sterile substrate comprising organic materials as a nutrient, and wherein the injection port is a sealed, one-way sterile inlet configured to introduce the mycelium spores and predetermined nutrients into the sterile substrate while preventing contamination;
a pressure relief valve coupled to the bioreactor configured to prevent pressure build-up from heating the sterile substrate;
a bioreactor microcontroller coupled to the bioreactor cartridge configured to monitor heat, oxygen, and nutrient levels;
a heating element coupled to the bioreactor microcontroller configured to heat the sterile substrate within the bioreactor cartridge to a predetermined temperature;
an air pump coupled to the bioreactor cartridge and to the bioreactor microcontroller configured to supply oxygenation of the mycelium spores;
an agitator located within the bioreactor cartridge and coupled to a motor driver coupled to the bioreactor microcontroller and configured to rotate at a predetermined speed to distribute heat, oxygen, and nutrients among the mycelium spores with predetermined parameters;
a cloud control system coupled to the bioreactor microcontroller and a remote server and configured to analyze known empirical data stored on the remote server related to cultivation of the mycelium spores to determine the predetermined speed, the predetermined parameters and the predetermined nutrients to produce specific harvest results and to make future automatic adjustments to the predetermined speed, the predetermined parameters and the predetermined nutrients of the bioreactor based on the specific harvest results; and
a bioreactor cloud data app coupled to a mobile device of the user configured to remotely monitor settings of the bioreactor, to allow the user to adjust the settings and to receive recommended settings based on the analysis of the cloud control system.

2. The portable and compact bioreactor for controlled cultivation of mycelium spores of claim 1, further comprising a processor coupled to the remote server configured to evaluate settings of the bioreactor cartridge by comparing the settings to known data and corresponding harvest results to identify recommended settings associated with predetermined harvest outcomes and further configured to transmit the recommended settings to the user.

3. The portable and compact bioreactor for controlled cultivation of mycelium spores of claim 1, wherein the cloud control system is further configured to collect and analyze settings and harvest results from other users as aggregated data to identify successful and unsuccessful patterns associated with outcomes and to automatically adjust settings of the bioreactor based on the aggregated data.

4. The portable and compact bioreactor for controlled cultivation of mycelium spores of claim 1, wherein the bioreactor microcontroller is further configured to automatically receive settings of the bioreactor cartridge and automatically adjust the settings to optimize cultivation results, using harvest data collected from another user identified by the user.

5. The portable and compact bioreactor for controlled cultivation of mycelium spores of claim 1, wherein the empirical data remote consists of historical bioreactor settings and corresponding harvest results and wherein the remote server is configured to analyze the empirical data to identify past configurations that produced desired outcomes.

6. The portable and compact bioreactor for controlled cultivation of mycelium spores of claim 1, further comprising a processor coupled to the remote server and configured to remotely and continuously monitor in real-time operational parameters of the bioreactor cartridge and compare the operational parameters against optimal historical settings and to dynamically adjust the operational parameters to maintain optimal harvest conditions.

7. The portable and compact bioreactor for controlled cultivation of mycelium spores of claim 1, further comprising a quick connect component with magnetic collars configured to mechanically lock the bioreactor cartridge in place within the bioreactor, to mechanically couple the bioreactor cartridge to the agitator and to electronically couple the bioreactor cartridge to the bioreactor microcontroller.

8. A portable and compact bioreactor for controlled cultivation of mycelium spores, comprising:
a bioreactor cartridge with an injection port, wherein the bioreactor cartridge is removably coupled to the bioreactor and contains a sterile substrate comprising organic materials as a nutrient, and wherein the injection port is a sealed, one-way sterile inlet configured to introduce the mycelium spores and predetermined nutrients into the sterile substrate while preventing contamination;
a pressure relief valve coupled to the bioreactor configured to prevent pressure build-up from heating the sterile substrate;
a bioreactor microcontroller coupled to the bioreactor cartridge configured to monitor heat, oxygen, and nutrient levels, wherein the bioreactor microcontroller is further configured to adjust parameter settings of the bioreactor cartridge based on analyzed optimal settings during cultivation that produce optimized harvest results;
a heating element coupled to the bioreactor microcontroller configured to heat the sterile substrate within the bioreactor cartridge to a predetermined temperature;
an air pump coupled to the bioreactor cartridge and to the bioreactor microcontroller configured to supply oxygenation of the mycelium spores;
an agitator located within the bioreactor cartridge and coupled to a motor driver coupled to the bioreactor microcontroller and configured to rotate at a predetermined speed to distribute heat, oxygen, and nutrients among the mycelium spores with predetermined parameters;
a cloud control system coupled to the bioreactor microcontroller configured to analyze empirical data related to cultivation of the mycelium spores to determine the predetermined speed, the predetermined parameters and the predetermined nutrients to produce specific harvest results and to make future automatic adjustments to the predetermined speed, the predetermined parameters and the predetermined nutrients based on the specific harvest results; and
a bioreactor cloud data app coupled to a mobile device of the user configured to remotely monitor settings of the bioreactor, to allow the user to adjust the settings and to receive recommended settings based on the analysis of the cloud control system.

9. The portable and compact bioreactor for controlled cultivation of mycelium spores of claim 8, further comprising a processor coupled to the remote server configured to evaluate settings of the bioreactor cartridge by comparing the settings to known data and corresponding harvest results to identify recommended settings associated with predetermined harvest outcomes and further configured to transmit the recommended settings to the user.

10. The portable and compact bioreactor for controlled cultivation of mycelium spores of claim 8, wherein the cloud control system is further configured to collect and analyze settings and harvest results from other users as aggregated data to identify successful and unsuccessful patterns associated with outcomes and to automatically adjust settings of the bioreactor based on the aggregated data.

11. The portable and compact bioreactor for controlled cultivation of mycelium spores of claim 8, wherein the bioreactor microcontroller is further configured to automatically receive settings of the bioreactor cartridge and automatically adjust the settings to optimize cultivation results, using harvest data collected from another user identified by the user.

12. The portable and compact bioreactor for controlled cultivation of mycelium spores of claim 8, wherein the empirical data remote consists of historical bioreactor settings and corresponding harvest results and wherein the remote server is configured to analyze the empirical data to identify past configurations that produced desired outcomes.

13. The portable and compact bioreactor for controlled cultivation of mycelium spores of claim, further comprising a processor coupled to the remote server and configured to remotely and continuously monitor in real-time operational parameters of the bioreactor cartridge and compare the operational parameters against optimal historical settings and to dynamically adjust the operational parameters to maintain optimal harvest conditions.

14. The portable and compact bioreactor for controlled cultivation of mycelium spores of claim, further comprising a quick connect component with magnetic collars configured to mechanically lock the bioreactor cartridge in place within the bioreactor, to mechanically couple the bioreactor cartridge to the agitator and to electronically couple the bioreactor cartridge to the bioreactor microcontroller.

15. A portable and compact bioreactor for controlled cultivation of mycelium spores, comprising:
a bioreactor cartridge with an injection port, wherein the bioreactor cartridge is removably coupled to the bioreactor and contains a sterile substrate comprising organic materials as a nutrient, and wherein the injection port is a sealed, one-way sterile inlet configured to introduce the mycelium spores and predetermined nutrients into the sterile substrate while preventing contamination;
wherein the bioreactor includes a front pivoting section of a top cover section to pivot open for the installation of a bioreactor cartridge and make connections to the microcontroller, air pump oxygenation system, agitator drive and positioned above the heating element;
a pressure relief valve coupled to the bioreactor configured to prevent pressure build-up from heating the sterile substrate;
a bioreactor microcontroller coupled to the bioreactor cartridge configured to monitor heat, oxygen, and nutrient levels, wherein the bioreactor microcontroller is further configured to adjust parameter settings of the bioreactor cartridge based on analyzed optimal settings during cultivation that produce optimized harvest results;
a heating element coupled to the bioreactor microcontroller configured to heat the sterile substrate within the bioreactor cartridge to a predetermined temperature;
an air pump coupled to the bioreactor cartridge and to the bioreactor microcontroller configured to supply oxygenation of the mycelium spores;
an agitator located within the bioreactor cartridge and coupled to a motor driver coupled to the bioreactor microcontroller and configured to rotate at a predetermined speed to distribute heat, oxygen, and nutrients among the mycelium spores with predetermined parameters;
a cloud control system coupled to the bioreactor microcontroller configured to analyze empirical data related to cultivation of the mycelium spores to determine the predetermined speed, the predetermined parameters and the predetermined nutrients to produce specific harvest results and to make future automatic adjustments to the predetermined speed, the predetermined parameters and the predetermined nutrients based on the specific harvest results; and
a bioreactor cloud data app coupled to a mobile device of the user configured to remotely monitor settings of the bioreactor, to allow the user to adjust the settings and to receive recommended settings based on the analysis of the cloud control system.

16. The portable and compact bioreactor for controlled cultivation of mycelium spores of claim 15, further comprising a processor coupled to the remote server configured to evaluate settings of the bioreactor cartridge by comparing the settings to known data and corresponding harvest results to identify recommended settings associated with predetermined harvest outcomes and further configured to transmit the recommended settings to the user.

17. The portable and compact bioreactor for controlled cultivation of mycelium spores of claim 15, wherein the cloud control system is further configured to collect and analyze settings and harvest results from other users as aggregated data to identify successful and unsuccessful patterns associated with outcomes and to automatically adjust settings of the bioreactor based on the aggregated data.

18. The portable and compact bioreactor for controlled cultivation of mycelium spores of claim 15, wherein the bioreactor microcontroller is further configured to automatically receive settings of the bioreactor cartridge and automatically adjust the settings to optimize cultivation results, using harvest data collected from another user identified by the user.

19. The portable and compact bioreactor for controlled cultivation of mycelium spores of claim 15, further comprising a processor coupled to the remote server and configured to remotely and continuously monitor in real-time operational parameters of the bioreactor cartridge and compare the operational parameters against optimal historical settings and to dynamically adjust the operational parameters to maintain optimal harvest conditions.

20. The portable and compact bioreactor for controlled cultivation of mycelium spores of claim 15, further comprising a quick connect component with magnetic collars configured to mechanically lock the bioreactor cartridge in place within the bioreactor, to mechanically couple the bioreactor cartridge to the agitator and to electronically couple the bioreactor cartridge to the bioreactor microcontroller.
